(19) United States
(12) Patent Application Publication
(10) Pub. No.: US 2025/0319292 A1
(43) Pub. Date: Oct. 16, 2025

(54) MICRONEEDLES AND METHODS FOR TREATING THE SKIN

(71) Applicants: THE BRIGHAM AND WOMEN'S HOSPITAL, INC., Boston, MA (US); MASSACHUSETTS INSTITUTE OF TECHNOLOGY, Cambridge, MA (US)

(72) Inventors: Natalie Artzi, Brookline, MA (US); Pere Dosta, Cambridge, MA (US); Nuria Puigmal, Brookline, MA (US); Zhabiz Solhjou, Boston, MA (US); Karim Yatim, Roxbury Crossing, MA (US); Jamil Azzi, Chestnut Hill, MA (US)

(21) Appl. No.: 18/278,903

(22) PCT Filed: Feb. 28, 2022
(86) PCT No.: PCT/US2022/018205

§ 371 Date: Aug. 25, 2023

Related U.S. Application Data
(60) Provisional application No. 63/154,690, filed on Feb. 27, 2021.
(60) Provisional application No. 63/154,850, filed on Mar. 1, 2021.

(51) Int. Cl. A61M 37/00

(52) U.S. Cl. CPC A61M 37/0015; A61M 2037/0023; A61M 2037/0061

(57) ABSTRACT

The present disclosure describes methods of treating a skin disorder in a subject in need thereof and methods of locally suppressing an immune response in a tissue of a subject in need thereof. The methods can include contacting a microneedle array comprising a plurality of microneedles with a skin surface of the subject, wherein the plurality of microneedles comprises: i) a degradable hyaluronic acid polymer comprising a disulfide bond, and ii) a therapeutic agent; and applying pressure on the microneedle array such that the plurality of microneedles penetrates the skin surface, thereby releasing the therapeutic agent beneath the skin surface while simultaneously capturing ISF for successive analysis.

### CLAIM OF PRIORITY

This application claims the benefit of U.S. Provisional Application Ser. No. 63/154,690, filed on Feb. 27, 2021 and claims the benefit of U.S. Provisional Application Ser. No. 63/154,850, filed on Mar. 1, 2021. The entire contents of the foregoing are hereby incorporated by reference.

### SEQUENCE LISTING

This application contains a Sequence Listing that has been submitted electronically as an ASCII text file named “Sequence_Listing.txt.” The ASCII text file, created on Feb. 28, 2022, is 3 kilobytes in size. The material in the ASCII text file is hereby incorporated by reference in its entirety.

### TECHNICAL FIELD

The present disclosure describes compositions comprising crosslinkable hyaluronic-based hydrogels including microneedle arrays. The disclosure also describes methods of delivering a therapeutic agent in a subject in need thereof using these compositions. The hydrogel compositions can include an amino-modified hyaluronic acid polymer comprising a disulfide bond. The methods of treating a skin disorder and methods of locally suppressing an immune response can include contacting a skin surface of the subject with the microneedle array compositions and applying pressure such that the tips of the plurality of microneedles perforate and/or penetrate the skin surface, thereby releasing the therapeutic agent in the tissue while simultaneously monitoring the immune cellular profile as a response to the therapy.

### BACKGROUND

Skin allografts serve as temporary dressing for patients suffering trauma after major burns due to their high immunogenicity and rejection by the immune system, requiring systemic immunosuppressive therapies that can lead to deleterious side effects. Systemic adoptive therapy with regulatory T cells (T_{regs}) has been proposed as a therapy to prevent skin allograft rejection and improving allograft survival after transplantation. However, the efficacy of systemic adoptive therapy with regulatory T_{regs} is limited by their short half-life and by the need to continuously deliver exogenous cytokines to maintain their immunosuppressive function. Thus, widespread translation of these therapies into clinical settings has been limited due to the premature clearance of T_{regs} from serum and their need for a favorable immune environment, including IL-2, to ensure their survival and phenotypic stability. Post-transplant immunosuppressive therapies are known to generate a hostile IL-2 depleted milieu for T_{regs} proliferation, but attempting to counteract the levels of IL-2 via systemic administration has been constrained by risks of infection, vascular leak syndrome, and the expansion of other proinflammatory cell counterparts such as natural killer (NK) cells. Previous studies have also shown increase in T_{regs} proliferation and population size in the spleen in response to systemic IL-2 delivery, while its effect on allograft survival has been limited compared to its broad range of side effects.

Recently, CCL22 has been proposed as a powerful candidate to mediate migration of T_{regs} to the site of inflammation and reestablish donor-specific tolerance in different transplant models including pancreatic islets allografts and vascularized allograft composites. Prompt recognition of rejection episodes is as critical as their management, particularly at early stages. However, current strategies to monitor skin transplant failure rely on gross observation and skin biopsies, which in addition to being invasive and biased due to the limited area that is being analyzed, becomes apparent late in the process, when intervention can no longer be effective. Thus, there is an unmet need for a therapeutic that can facilitate efficient immune cell delivery and tissue cell sampling to enhance an immune tolerogenic environment and potentially monitor changes in the tissue inflammatory state.

### SUMMARY

Certain aspects of the present disclosure are directed to methods of treating a skin disorder in a subject in need thereof, the method comprising: contacting a microneedle array comprising a plurality of microneedles with a skin surface of the subject, wherein the plurality of microneedles comprises: i) a degradable hyaluronic acid polymer comprising a disulfide bond coupled to a terminal amine group, and ii) an immunosuppressor and/or an immunoregulator; and applying pressure on the microneedle array such that the plurality of microneedles penetrates the skin surface, thereby releasing the immunosuppressor and/or the immunoregulator beneath the skin surface, wherein the degradable hyaluronic acid polymer preferably comprises the following chemical structure:

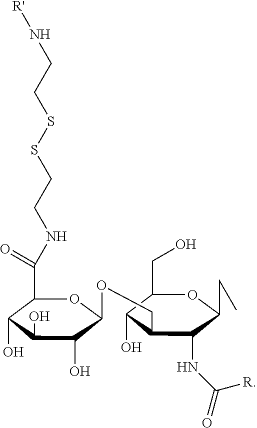

In some embodiments, the skin disorder is diffuse systemic scleroderma, atopic dermatitis, cutaneous lupus erythematosus, alopecia areata, alopecia totalis, alopecia universalis, androgenetic alopecia, vitiligo, psoriasis, a burn, or any combination thereof.

Certain aspects of the present disclosure are directed to methods of locally regulating an immune response in a tissue of a subject in need thereof, the method comprising: contacting a microneedle array comprising a plurality of microneedles with a skin surface of the subject, wherein the plurality of microneedles comprises: i) a degradable hyaluronic acid polymer comprising a disulfide bond, and ii) an immunosuppressor and/or an immunoregulator; and applying pressure on the microneedle array such that the plurality of microneedles penetrates the skin surface, thereby releasing the immunosuppressor and/or the immunoregulator beneath the skin surface in the tissue.

In some embodiments, the tissue is a burned tissue, an autograft, a split-thickness skin graft, a full-thickness skin graft, an allograft, a homograft, a xenograft, a meshed graft, a sheet graft, or any combination thereof. In some embodiments, the immunosuppressor and/or the immunoregulator comprises a cell, a chemokine, a cytokine, an anti-inflammatory agent, an antibody, or any combination thereof. In some embodiments, the cell is a T cell, B cell, 20 natural killer cell, dendritic cell, macrophage, or any combination thereof. In some embodiments, the chemokine comprises C-C motif chemokine 22 (CCL22). In some embodiments, the cytokine comprises IL-7, IL-2, IL-10, TGF-β, IL-33, IFN-alpha, IFN-β or any combination thereof.

In some embodiments, the antibody comprises an anti-CD3 monoclonal antibody, an anti-IL-6 monoclonal antibody, an anti-CD28 monoclonal antibody, an anti-CD52 monoclonal antibody, or any combination thereof. In some embodiments, the anti-inflammatory agent comprises a corticosteroid, a non-steroidal anti-inflammatory drug (NSAIDs), an anti-inflammatory cytokine, a cytokine antagonist, an immunosuppressant, an mTOR inhibitor, rapamycin, or any combination thereof. In some embodiments, the releasing the immunosuppressor and/or the immunoregulator beneath the skin surface does not elicit a systemic response. In some embodiments, the methods further comprise sampling an interstitial fluid using the plurality of microneedles.

In some embodiments, the sampling comprises absorbing the interstitial fluid with the plurality of microneedles. In some embodiments, the interstitial fluid comprises a biomarker and/or a cell. In some embodiments, the methods further comprise depleting an unwanted immune cell by recruiting the unwanted cells with the immunosuppressor and/or the immunoregulator. In some embodiments, each microneedle of the plurality of microneedles is a degradable microneedle configured to be degraded upon exposure to a reducing agent. In some embodiments, the disulfide bond is configured to be cleaved upon exposure to the reducing agent. In some embodiments, the reducing agent is glutathione, dithiothreitol, or beta-mercaptoethanol. In some embodiments, the reducing agent is tris(2-carboxyethyl)phosphine (TCEP). In some embodiments, the disulfide bond is configured to be cleaved by exposure to about 1 mM to about 100 mM of TCEP.

In some embodiments, the hyaluronic acid polymer is a crosslinked hyaluronic acid polymer. In some embodiments, the crosslinked hyaluronic acid polymer is chemically crosslinked via a chemical crosslinker. In some embodiments, the chemical crosslinker comprises polyethylene glycol (PEG) further comprising a succinimidyl functional group In some embodiments, the PEG is an 8-arm PEG. In some embodiments, the PEG has a molecular weight ranging from about 10 kDa to about 200 kDa. In some embodiments, the chemical crosslinker comprises a first PEG having a molecular weight of about 40 kDa and a second PEG having a molecular weight of about 10 kDa. In some embodiments, the chemical crosslinker comprises the first PEG and the second PEG at a ratio of about 0:100 to about 100:0. In some embodiments, each microneedle of the plurality of microneedles has a height of about 100 μm to about 1,500 μm and a base having a radius of about 100 μm to about 1,500 μm. In some embodiments, each microneedle has a height of about 600 μm and a base having a radius of about 150 μm. In some embodiments, the plurality of microneedles project from a polymeric, biodegradable substrate. In some embodiments, the polymeric, biodegradable substrate comprises poly(D,L-lactide-co-glycolide) polymer.

In some embodiments, each microneedle of the plurality of microneedles has a swelling ratio ranging from about 800% to about 1800% within at least about 15 minutes of exposure to an aqueous medium. In some embodiments, each microneedle of the plurality of microneedles is a porous hydrogel.

The terms “subject” or “patient” as used herein refer to any mammal (e.g., a human or a veterinary subject, e.g., a dog, cat, horse, cow, goat, sheep, mouse, rat, or rabbit) to which a composition or method of the present disclosure may be administered, e.g., for experimental, diagnostic, prophylactic, and/or therapeutic purposes. The subject may seek or need treatment, require treatment, is receiving treatment, will receive treatment, or is under care by a trained professional for a particular disease or condition.

The term “composition” as used herein can refer to a microneedle array composition, a precursor composition (e.g., a composition before crosslinking polymerization), and/or a hydrogel composition (e.g., a hydrogel composition after crosslinking polymerization), as provided by the corresponding context of the disclosure.

As used herein, the singular forms “a,” “an,” and “the” include plural referents unless the context clearly dictates otherwise. Thus, for example, reference to “a micelle” includes mixtures of micelles, reference to “a micelle” includes mixtures of two or more such micelles, and the like.

As used herein, the term “therapeutic agent” is any molecule or atom that is encapsulated, conjugated, fused, dispersed, embedded, mixed, or otherwise affixed to any of the compositions described herein and is useful for a disease therapy.

As used herein, the term “payload” refers to an agent delivered by a chemically-modified hydrogel composition described herein (e.g., chemically-modified hyaluronic acid hydrogel microneedles or other chemically-modified hyaluronic acid hydrogel compositions).

By the term “nanoparticle” is meant an object that has a diameter between about 2 nm to about 200 nm (e.g., between 10 nm and 200 nm, between 2 nm and 100 nm, between 2 nm and 40 nm, between 2 nm and 30 nm, between 2 nm and 20 nm, between 2 nm and 15 nm, between 100 nm and 200 nm, and between 150 nm and 200 nm). Non-limiting examples of nanoparticles include the nanoparticles described herein. Additional examples of nanoparticles are known in the art.

By the term “nucleic acid” is meant any single- or double-stranded polynucleotide (e.g., DNA or RNA having a semi-synthetic or a synthetic origin). The term nucleic acid includes oligonucleotides containing at least one modified nucleotide (e.g., containing a modification in the base and/or a modification in the sugar) and/or a modification in the phosphodiester bond linking two nucleotides. Exemplary nucleic acids for use in accordance with the present disclosure include, but are not limited to, one or more of DNA, RNA, hybrids thereof, RNAi-inducing agents, RNAi agents, siRNAs, shRNAs, miRNAs, mRNAs, antisense RNAs, ribozymes, catalytic DNA, RNAs that induce triple helix formation, aptamers, vectors, and the like. Additional non-limiting examples of nucleic acids are described herein and are known in the art.

As used herein, the expression “pharmaceutically acceptable” applies to a composition which contains composition ingredients that are compatible with other ingredients of the composition as well as physiologically acceptable to the recipient (e.g., a mammal such as a human) without the resulting production of excessive undesirable and unacceptable physiological effects or a deleterious impact on the mammal being administered the pharmaceutical composition. A composition as described herein can comprise one or more carriers, useful excipients, and/or diluents.

As used herein, the term “hydrogel” refers to a polymeric material having a three-dimensional physical or covalently cross-linked networks that have an affinity for an aqueous medium and are able to absorb a large amount of water while maintaining a semisolid morphology (e.g., they do not normally dissolve in the aqueous medium unless they are triggered to do so).

As used herein, the term “aqueous medium” as used herein refers to water or a solution based primarily on water such as phosphate-buffered saline (PBS), or water containing one or more salts dissolved therein.

As used herein, the term “crosslink” refers to an interconnection between polymer chains via chemical bonding, such as, but not limited to, covalent bonding, ionic bonding, or affinity interactions that are caused by a chemical composition (e.g., a crosslinker).

As used herein, the term “biodegradable” refers to a substance which may be broken down by microorganisms, or which spontaneously breaks down over a relatively short time (within about 14 days to about 6 months) when exposed to environmental conditions commonly found in nature. For example, the compositions described herein may be degraded by a reducing agent (e.g., TCEP) that is contacted with the composition.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. Furthermore, the use of the term “about,” as used herein, refers to an amount that is near the stated amount by about 10%, 5%, or 1%, including increments therein. For example, “about” can mean a range including the particular value and ranging from 10% below that particular value and spanning to 10% above that particular value.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

Where values are described in the present disclosure in terms of ranges, endpoints are included. Furthermore, it should be understood that the description includes the disclosure of all possible sub-ranges within such ranges, as well as specific numerical values that fall within such ranges irrespective of whether a specific numerical value or specific sub-range is expressly stated.

Various embodiments of the features of this disclosure are described herein. However, it should be understood that such embodiments are provided merely by way of example, and numerous variations, changes, and substitutions can occur according to those skilled in the art without departing from the scope of this disclosure. It should also be understood that various alternatives to the specific embodiments described herein are also within the scope of this disclosure.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features and advantages of the invention will be apparent from the following detailed description and figures, and from the claims.

### DESCRIPTION OF DRAWINGS

FIGS. 1A-1C show the design of an HA-based MN platform. FIG. 1A illustrates the use of a novel HA-based MN platform that allows for: (1) delivery of drugs and (2) in situ biomarkers and cell sampling for monitoring. FIG. 1B illustrates the HA-based MN fabrication process. HA-based MNs were fabricated by casting an aqueous amine-modified HA (HA-SS—NH₂) solution into the PDMS mold by centrifugation and crosslinked using the NHS-terminated 8-arm PEG crosslinker. Chemokines were loaded and a PLGA back layer was added (top scheme). Chemical structure of the HA-SS—NH₂ crosslinked with NHS-terminated 8-arm PEG forming a digestible HA hydrogel through a disulfide bond (bottom scheme). FIG. 1C illustrates how the degradation of the MNs under reducing conditions enables facile recovery of retrieved cells. Disulfide bonds of the HA-based MNs are cleaved with 10 mM TCEP.

FIGS. 2A-2E show HA-modified MNs present high swelling capacity, robust mechanical properties and on-demand degradation. FIG. 2A is a graph showing the swelling rate of the HA-based hydrogels composed of amine-modified HA polymer crosslinked with NHS-terminated 8-arm PEG crosslinkers differing in molecular weight. FIG. 2B is an image of stained skin graft following ex vivo HA-SS—NH₂-derived MN application confirms skin penetration. Scale bar=2 mm. FIG. 2C is an image showing the mouse skin after in vivo administration of hydrogel MNs. Scale bar=2 mm. FIG. 2D is a graph showing on-demand digestion of hydrogel disks using varying concentrations of the reducing agent, TCEP. FIG. 2E is a group of optical microscopy images of hydrogel-based MNs before (top) and after (bottom) digestion under reducing conditions. Scale bar=500 μm. Data is represented as mean±s.d. (n=3).

FIGS. 3A-3D shows the characterization of MN-based platform for immune cell sampling in vitro. FIG. 3A is a graph showing the release of the IL-2 (drug model) from the HA-based hydrogel MNs quantified by tracking the fluorescence signal of the labeled IL-2 over time. FIG. 3B is a graph showing the analysis of the MNs loading capacity by means of fluorescence quantification (plotted as initially loaded mass of IL-2 versus recovered mass of IL-2, R2=0.9987). FIG. 3C is a graph showing the comparison of T_{regs} migration as a function of CCL22 concentration when soluble or MN-loaded. FIG. 3D is a group of images showing the analyte recovery (RhoB) from mimetic skins with HA-derived MNs. FIG. 3E is a graph showing the detected RhoB concentration compared to the real RhoB concentration in HA-based MNs, R²=0.9929. FIG. 3F is a graph showing the recovered immune cells from digested HA-based MNs as quantified by flow cytometry. Data is represented as mean±s.d. (n=3). Multiple comparisons among groups were determined using either one-way ANOVA followed by a post-hoc test or non-parametric t test (Mann-Whitney) when applicable. P-value: ns=not significant, *p<0.05, **p<0.01.

FIGS. 4A-4E shows the delivery of HA-based MNs loaded with CCL22 and IL-2 to skin allografts results in increased T_{regs} recruitment. FIG. 4A is a schematic illustrating the study design of skin allograft transplant model. A 10 mm×15 mm skin patch from a BALB/cJ mouse was transplanted onto the dorsal trunk of a Rag^{-/-} mouse on C57BL/6 background. On day 6 post-transplant (shown as “day 0”), T lymphocytes were adoptively transferred, and on day 7 post-transplant (shown as “day 1”), MNs were applied consecutively for 5 days. At day 7 post-adoptive, T lymphocyte transfer, skin was harvested and analyzed by RT-PCR. FIG. 4B is a graph showing the FOXP3 to CD3 expression ratio.

FIG. 4C is a graph the fold change in IL-6 gene expression as quantified by RT-PCR. FIG. 4D is a graph showing the quantification, by flow cytometry, of the number of T_{regs} (FOXP3; CD4⁺) per million of splenocytes following CCL22+IL-2 administration via MNs compared to empty MNs as control. FIG. 4E is a set of graphs showing representative flow-cytometry-dot-plots of the number of T_{regs} (FOXP3⁺; CD4⁺) in the splenocyte proliferation study. Data are represented as mean±s.e.m. (n=4-9). Multiple comparisons among groups were determined using either one-way ANOVA followed by a post-hoc or non-parametric t test (Mann-Whitney) when applicable. P-value: ns=not significant, *p<0.05, **p<0.01.

FIGS. 5A-5C show the T_{regs} homing process monitoring using HA-based MNs. FIG. 5A is a schematic illustrating cell sampling using HA-based MNs or a skin graft biopsy. Cells were analyzed by flow cytometry. FIGS. 5B and 5C are graphs showing CD8⁺, CD4⁺, and FOXP3⁺ representative flow cytometry dot plots from retrieved ISF and skin allograft, respectively.

FIG. 6 is a schematic illustrating the simultaneous delivery of exogenous cytokines and sampling of regulatory T cells in interstitial fluid using the MNs disclosed herein.

### DETAILED DESCRIPTION

The compositions described herein include degradable, chemically crosslinkable hyaluronic acid-based hydrogels including microneedle arrays. In some examples, the compositions described herein are used for targeted, transdermal drug delivery. Methods of treating skin conditions and method of locally suppressing an immune response using these compositions are also provided herein. Some embodiments of the compositions and methods described herein may provide one or more of the following advantages.

Certain embodiments of the present disclosure include biocompatible, crosslinkable hyaluronic acid-based hydrogels. As discussed above, there is currently an unmet need for a capable of therapeutic and diagnostic applications. The compositions and methods of the present disclosure address this need. For example, in some embodiments, the microneedle array compositions and methods described herein can deliver a therapeutic agent payload while simultaneously sampling an interstitial fluid in a tissue, thereby offering an opportunity for diagnosis and monitoring of the response to therapy to further personalize it to the needs of the patient. In some embodiments, the chemical structure of the biocompatible polymer together with a crosslinker forms highly swellable hydrogel microneedles. In some embodiments, upon retrieval, the microneedles can be digested ex vivo by adding a reductive agent in less than 5 minutes for subsequent recovery and analysis of the biomarkers. Hence, the microneedle array compositions disclosed herein are a platform that can provide a quick readout of the patient state to intercept the disease and treat it prior to reaching irreversible states.

Some embodiments described herein may provide a theranostic microneedle platform using hyaluronic acid to deliver different types of drugs while enabling simultaneous sampling of the soluble and cellular fraction of the ISF. In some embodiments, the HA-based microneedles offer provide non-invasive delivery of various therapeutic cargos while retrieving biomarkers present in ISF. In some embodiments, the various therapeutic cargoes include a molecules of a wide range of molecular weights in the nano- and micro-scales, as outlined in detail elsewhere herein.

In some embodiments, the microneedle-based delivery methods described herein allow precise local delivery of a therapeutic agent within the skin, thereby reducing the off-target, side effects associated with systemic drug delivery. In some embodiments, the microneedle array compositions and methods of the disclosure are non-invasive and pain-free, thereby facilitating high patient compliance while minimizing the risk of infections. In some embodiments, the microneedle array compositions and methods of the disclosure can be used for diagnostic purposes serving as a non-invasive tool for interstitial fluid (ISF) extraction. ISF is a rich source of biomarkers that has been confirmed in recent clinical trials to intimately correlate with those biomarkers present in plasma and other conventional sources. Therefore, in some embodiments, ISF monitoring using the microneedle array compositions and methods of the disclosure can inform on tissue physiology by sampling both soluble biomarkers and cells and in turn report on the patient physiological state.

Some embodiments described herein may provide a hydrogel composition that may have tunable properties. For example, the swelling ratios, mechanical strength, degradation, and drug release kinetics of the hydrogel compositions described herein may be optimized by varying the molecular weight and concentration of one or more crosslinkers. Furthermore, in some embodiments, the hydrogel compositions described herein have on-demand degradation that is controlled by exposure to a reducing agent. Thus, the in some embodiments, the compositions and methods of the disclosure may provide a flexible drug delivery platform that can be optimized for various applications.

In some embodiments, the microneedle arrays of the disclosure can locally deliver immunomodulators and simultaneously sample immune cells in interstitial fluid to monitor the response to a therapy. In some embodiments, the cells can be retrieved from the microneedles for downstream analysis by degrading the hyaluronic acid using a reducing agent. In some embodiments, the microneedle arrays of the disclosure can be used to attract immune cells (e.g., T_{regs}) to a target tissue site and promote their expansion. In some embodiments, the microneedle arrays of the disclosure can be used to locally suppress an immune response in a tissue transplant (e.g., an allograft). Moreover, the microneedle arrays described herein can help regulate the immune system locally and facilitate the monitoring of the efficacy of immunotherapy after transplantation of a tissue (e.g., a skin tissue).

### Microneedle (MN) Arrays

### Structure of MN Arrays

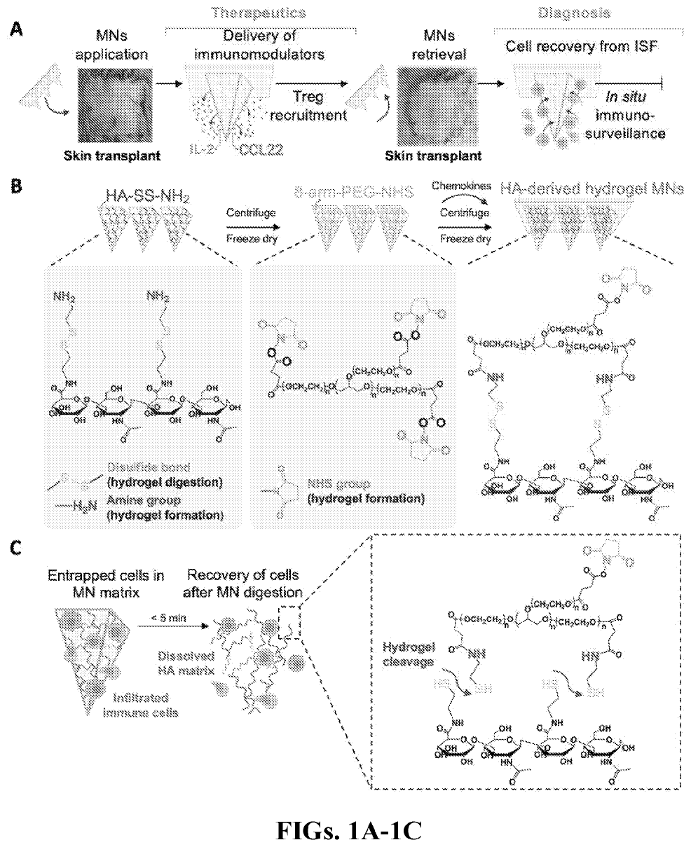

The present disclosure features microneedle array compositions that can include a plurality of microneedles projecting from a substrate. In some embodiments, the plurality of microneedles of the disclosure are degradable, porous, drug-eluting hydrogels that may facilitate the delivery of a drug through the structural barriers of tissues (e.g., skin) and/or sampling of interstitial fluid within a tissue (e.g., a skin tissue) upon application, as illustrated in FIG. 1A. For example, the microneedle arrays of the disclosure can be applied to a skin surface to deliver a therapeutic agent directly to an injured site, a disease site, and/or a transplant site in the skin of a patient. To this end, the microneedle array compositions described herein can further include one or more therapeutic agents.

In some embodiments, the microneedle array comprises a substrate from which the plurality of microneedles project therefrom. In some embodiments, the substrate is configured to be an anchor for microneedle array administration and retrieval. For example, when in use, the user (e.g., a clinician) can grasp the microneedle array by a substrate surface or an edge of the surface prior to applying the microneedle array on a skin surface of the patient. In some embodiments, the substrate is a substantially planar surface. In some embodiments, the substrate is a backing layer. In some embodiments, the substrate is a polymeric substrate. In some embodiments, the substrate is a biodegradable substrate. In some embodiments, the substrate is a non-biodegradable substrate. In some embodiments, the substrate comprises a non-soluble polymer. In some embodiments, the substrate comprises poly(D,L-lactide-co-glycolide) (PLGA) polymer. In some embodiments, the substrate comprises a water-soluble polymer. In some embodiments, the substrate comprises poly(vinyl alcohol) (PVA). In some embodiments, the substrate comprises polyethylene glycol diacrylate. Non-limiting examples of polymers that can be used to prepare a substrate include poly(caprolactone), poly(ethylene) glycol, poly(vinyl) pyrrolidone, poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactides (PLA), polyglycolides (PGA), polyanhydrides, polyorthoesters, polycyanoacrylate polycaprolactone, cellulose, lignin, alginate, chitosan, starch, or any combination thereof.

In some embodiments, each microneedle of the plurality of microneedles comprises a penetrating tip and a base that is integrally connected with the substrate. In some embodiments, each microneedle of the plurality of microneedles comprises a penetrating tip and a base that is removably connected with the substrate (e.g., each microneedle can be configured to be detached from the substrate via a trigger mechanism such as the dissolution of the substrate). In some embodiments, each microneedle has an elongate body having a proximal end and a distal end. In some embodiments, the elongate body generally tapers from the proximal end, near the base, to the distal end, near the penetrating tip. In some embodiments, each microneedle has a pyramidal or conical shape such that the microneedles taper to a point or a tip that is configured to perforate and/or penetrate a skin surface. The dimensions and geometry of the microneedles can vary as desired.

In some embodiments, each microneedle has a height ranging from about 100 μm to about 1,500 μm (e.g., about 100 μm to about 600 μm, about 200 μm to about 600 μm, about 300 μm to about 600 μm, about 400 μm to about 600 μm, about 500 μm to about 600 μm, about 600 μm to about 700 μm, about 600 μm to about 800 μm, about 600 μm to about 900 μm, about 600 μm to about 1000 μm, about 600 μm to about 1100 μm, about 600 μm to about 1200 μm, about 600 μm to about 1300 μm, about 600 μm to about 1400 μm, or about 600 μm to about 1500 μm). In some embodiments, each microneedle has a height of about 600 μm. The height of each microneedle can be measured from the base at the proximal end of the microneedle to the tip at the distal end of the microneedle. In some embodiments, each microneedle has a height that is sufficient to penetrate the stratum corneum and pass into the epidermis and/or the dermis.

In some embodiments, each microneedle has a base (e.g., a circular base or a rectangular base) having a width (e.g., a radius or rectangular width) ranging from about 100 μm to about 10,000 μm (e.g., about 100 μm to about 150 μm, about 125 μm to about 150 μm, about 150 μm to about 200 μm, about 150 μm to about 300 μm, about 150 μm to about 400 μm, about 150 μm to about 500 μm, about 150 μm to about 600 μm, about 150 μm to about 700 μm, about 150 μm to about 800 μm, about 150 μm to about 900 μm, about 150 μm to about 1000 μm, about 150 μm to about 1100 μm, about 150 μm to about 1200 μm, about 150 μm to about 1300 μm, about 150 μm to about 1400 μm, or about 150 μm to about 1500 μm, about 150 μm to about 5000 μm, about 150 μm to about 7,500 μm, about 150 μm to about 10,000 μm, about 1500 μm to about 5000 μm, about 1500 μm to about 7,500 μm, or about 1500 μm to about 10,000 μm). In some embodiments, each microneedle has a circular base having a radius of about 150 μm. In some embodiments, each microneedle has a rectangular base having a width of about 300 μm. The radius of a circular base of each microneedle can be defined as the distance from the center of the circular base to an edge of the circular base. The width of a rectangular base of each microneedle can be defined as the distance from a first edge of the rectangular base to a second, directly opposing edge of the rectangular base.

In some embodiments, each microneedle has a tip width ranging from about 1 μm to about 500 μm (e.g., about 1 μm to about 5 μm, about 1 μm to about 10 μm, about 1 μm to about 15 μm, about 1 μm to about 20 μm, about 1 μm to about 25 μm, about 1 μm to about 30 μm, about 5 μm to about 10 μm, about 5 μm to about 15 μm, about 5 μm to about 20 μm, about 5 μm to about 25 μm, about 5 μm to about 30 μm, about 10 μm to about 15 μm, about 10 μm to about 20 μm, about 10 μm to about 25 μm, about 10 μm to about 30 μm, 15 μm to about 20 μm, about 15 μm to about 25 μm, about 15 μm to about 30 μm, about 20 μm to about 25 μm, about 20 μm to about 30 μm, about 25 μm to about 30 μm, about 1 μm to about 300 μm, about 1 μm to about 400 μm, about 1 μm to about 500 μm, about 300 μm to about 400 μm, about 300 μm to about 500 μm, about 10 μm to about 300 μm, about 50 μm to about 300 μm, about 100 μm to about 300 μm, about 150 μm to about 300 μm, or about 250 μm to about 300 μm). In some embodiments, each microneedle has a tip width of about 300 μm.

In some embodiments, the microneedle array can have any suitable shape or size. In some embodiments, the microneedle array is an array of microneedles having dimensions ranging from about 5×5 to about 20×20 (e.g., about 5×5 to about 11×11, about 6×6 to about 11×11, about 7×7 to about 11×11, about 8×8 to about 11×11, about 9×9 to about 11×11, about 10×10 to about 11×11, about 11×11 to about 15×15, about 11×11 to about 20×20. In some embodiments, the microneedle array is an 11×11 array of microneedles. In some embodiments, a density of the plurality of microneedles of the microneedle array can range between about 100 microneedles/cm² to about 1000 microneedles/cm² or more (e.g., about 100 microneedles/cm² to about 500 microneedles/cm², about 100 microneedles/cm² to about 600 microneedles/cm², about 100 microneedles/cm² to about 700 microneedles/cm², about 100 microneedles/cm² to about 800 microneedles/cm², about 900 microneedles/cm² to about 500 microneedles/cm², about 100 microneedles/cm² to about 950 microneedles/cm², about 500 microneedles/cm² to about 600 microneedles/cm², about 500 microneedles/cm² to about 700 microneedles/cm², about 500 microneedles/cm² to about 800 microneedles/cm², about 500 microneedles/cm² to about 900 microneedles/cm², about 500 microneedles/cm² to about 1000 microneedles/cm², or more). In some embodiments, a density of the plurality of microneedles of the microneedle array is about 500 microneedles/cm².

In some embodiments, the microneedle array can be arranged in a variety of ways. In some embodiments, the microneedle array can be arranged with a tip-to-tip spacing between microneedles ranging from about 50 μm to about 1000 μm (e.g., about 50 μm to about 600 μm, about 100 μm to about 600 μm, about 200 μm to about 600 μm, about 300 μm to about 600 μm, about 400 μm to about 600 μm, about 500 μm to about 600 μm, about 600 μm to about 700 μm, about 600 μm to about 800 μm, about 600 μm to about 900 μm, or about 600 μm to about 1000 μm). In some embodiments, the microneedle array can be arranged with a tip-to-tip spacing between microneedles of about 600 μm.

### Functionalized HA Polymer

Hyaluronic acid (HA) is a viscoelastic, biocompatible, biodegradable, non-toxic, and non-immunogenic natural linear polysaccharide with high water affinity. HA is known to play a role in the regeneration and reconstruction of soft tissues. In some embodiments, a chemically-modified HA can be included in the microneedle array compositions of the present disclosure. In some embodiments, the entire structure of each microneedle (e.g., from the base to the tip) is composed of the chemically-modified HA described herein. In some embodiments, the chemically modified HA can be an amino-modified hyaluronic acid comprising a disulfide bond (HA-SS—NH₂).

In some embodiments, the HA polymer that is chemically modified has a molecular weight ranging from about 0.5 kilodalton (kDa) to about 20,000 kDa (e.g., about 0.5 kDa to about 60 kDa, about 1 kDa to about 60 kDa, about 5 kDa to about 60 kDa, about 10 kDa to about 60 kDa, about 20 kDa to about 60 kDa, about 30 kDa to about 60 kDa, about 40 kDa to about 60 kDa, about 50 kDa to about 60 kDa, about 60 kDa to about 75 kDa, about 60 kDa to about 100 kDa, about 60 kDa to about 200 kDa, about 60 kDa to about 300 kDa, about 60 kDa to about 400 kDa, about 60 kDa to about 500 kDa, about 60 kDa to about 600 kDa, about 60 kDa to about 700 kDa, about 60 kDa to about 800 kDa, about 60 kDa to about 900 kDa, about 60 kDa to about 1000 kDa, about 60 kDa to about 2000 kDa, about 60 kDa to about 3000 kDa, about 60 kDa to about 4000 kDa, about 60 kDa to about 5000 kDa, about 60 kDa to about 6000 kDa, about 60 kDa to about 7000 kDa, about 60 kDa to about 8000 kDa, about 60 kDa to about 9000 kDa, about 60 kDa to about 10,000 kDa, about 60 kDa to about 11,000 kDa, about 60 kDa to about 12,000 kDa, about 60 kDa to about 13,000 kDa, about 60 kDa to about 14,000 kDa, about 60 kDa to about 15,000 kDa, about 60 kDa to about 16,000 kDa, about 60 kDa to about 17,000 kDa, about 60 kDa to about 18,000 kDa, about 60 kDa to about 19,000 kDa, about 60 kDa to about 20,000 kDa, about 1000 kDa to about 5000 kDa, about 1000 kDa to about 10,000 kDa, about 1000 kDa to about 15,000 kDa, about 1000 kDa to about 20,000 kDa.

In some embodiments, the chemically-modified HA polymer includes one or more side chains including one or more functional groups (e.g., a disulfide group and an amine group), each side chain having a length ranging from about 3 carbon atoms to about 100 carbon atoms (e.g., about 3 carbon atoms to about 4 carbon atoms, about 3 carbon atoms to about 5 carbon atoms, about 3 carbon atoms to about 6 carbon atoms, about 3 carbon atoms to about 7 carbon atoms, about 3 carbon atoms to about 8 carbon atoms, about 3 carbon atoms to about 9 carbon atoms, about 3 carbon atoms to about 10 carbon atoms, about 3 carbon atoms to about 15 carbon atoms, about 3 carbon atoms to about 20 carbon atoms, about 3 carbon atoms to about 25 carbon atoms, about 3 carbon atoms to about 30 carbon atoms, about 3 carbon atoms to about 35 carbon atoms, about 3 carbon atoms to about 40 carbon atoms, about 3 carbon atoms to about 45 carbon atoms, about 3 carbon atoms to about 50 carbon atoms, about 3 carbon atoms to about 55 carbon atoms, about 3 carbon atoms to about 60 carbon atoms, about 3 carbon atoms to about 65 carbon atoms, about 3 carbon atoms to about 70 carbon atoms, about 3 carbon atoms to about 75 carbon atoms, about 3 carbon atoms to about 80 carbon atoms, about 3 carbon atoms to about 85 carbon atoms, about 3 carbon atoms to about 90 carbon atoms, about 3 carbon atoms to about 95 carbon atoms, about 3 carbon atoms to about 99 carbon atoms, about 10 carbon atoms to about 50 carbon atoms, about 50 carbon atoms to about 100 carbon atoms, or more).

In some embodiments, the chemically-modified HA polymer includes one or more side chains having a same chain length. In some embodiments, the chemically-modified HA polymer includes one or more side chains having a different chain length. In some embodiments, the one or more side chains include an amine group and a disulfide group. In some embodiments, the one or more side chains include an amine group. In some embodiments, the one or more side chains include a disulfide group. In some embodiments, the chemically modified hyaluronic acid comprises one or more side functional groups. In some embodiments, the chemically modified hyaluronic acid comprises one or more side chains including one or more side functional groups. In some embodiments, the side functional groups comprise one or more disulfide groups, thiol groups, urea groups, carboxylic ester groups, carboxylic acid groups, carboxylic acid salts, latent carboxylic acid groups, quaternary amine groups, tertiary amine groups, secondary amine groups, primary amine groups, azides, alkynes, poly(alkylene ether) groups, and any combinations thereof.

In some embodiments, the plurality of microneedles comprises a degradable hyaluronic acid polymer comprising a disulfide bond. In some embodiments, the degradable hyaluronic acid polymer is cross-linkable. In some embodiments, the degradable hyaluronic acid polymer is an amino-modified hyaluronic acid prior to being crosslinked (e.g., in a precursor state). As described in Example 1, the hyaluronic acid polymer can be synthesized by activating sodium hyaluronate with N-(3-(dimethylamino)propyl)carbodiimide (EDC) and N-hydroxysuccinimide (NHS). Next, as described in Example 1, the activated hyaluronic acid can be mixed with cysteamine dihydrochloride and reacted for about 12 hours. In some embodiments, the activated hyaluronic acid can be mixed with cysteamine dihydrochloride at a ratio ranging from about 1:1 to about 1:20 (e.g., about 1:1 to about 1:10, about 1:2 to about 1:10, about 1:3 to about 1:10, about 1:4 to about 1:10, about 1:5 to about 1:10, about 1:6 to about 1:10, about 1:7 to about 1:10, about 1:8 to about 1:10, about 1:9 to about 1:10, about 1:10 to about 1:15, or about 1:10 to about 1:20). In some embodiments, the activated hyaluronic acid can be mixed with excess cysteamine dihydrochloride. The addition of cysteamine dihydrochloride to the activated hyaluronic acid functionalizes the hyaluronic acid with a terminal amino group and the disulfide bond, as shown in FIG. 1B.

In some embodiments, the plurality of microneedles comprises a degradable hyaluronic acid polymer having an on-demand degradation dependent upon the cleavage of the disulfide bond. For example, in some embodiments, the degradable hyaluronic acid polymer can have a degradation profile that is controlled by the addition of a reducing agent that cleaves the disulfide bond. In some embodiments, each microneedle of the plurality of microneedles is a degradable microneedle configured to be degraded upon exposure to the reducing agent. In some embodiments, the disulfide bond is configured to be cleaved upon exposure to the reducing agent. In some embodiments, the reducing agent is tris(2-carboxyethyl)phosphine (TCEP). In some embodiments, the reducing agent is glutathione, dithiothreitol, beta-mercaptoethanol, or any combination thereof.

In some embodiments, the disulfide bond is configured to be cleaved by exposure to about 1 millimolar (mM) to about 100 mM of TCEP (e.g., about 1 mM to about 10 mM, about 2 mM to about 10 mM, about 3 mM to about 10 mM, about 4 mM to about 10 mM, about 5 mM to about 10 mM, about 6 mM to about 10 mM, about 7 mM to about 10 mM, about 8 mM to about 10 mM, about 9 mM to about 10 mM, about 10 mM to about 20 mM, about 10 mM to about 30 mM, about 10 mM to about 40 mM, about 10 mM to about 50 mM, about 10 mM to about 60 mM, about 10 mM to about 70 mM, about 10 mM to about 80 mM, about 10 mM to about 90 mM, about 10 mM to about 100 mM). In some embodiments, the disulfide bond is configured to be cleaved by exposure to about 10 mM of TCEP.

Generally, a hydrogel may be formed by using at least one, or one or more types of hydrogel precursors, and setting or solidifying the one or more types of hydrogel precursors in an aqueous solution to form a three-dimensional network, wherein formation of the three-dimensional network may cause the one or more types of hydrogel precursors to gel. As used herein, the term “hydrogel precursor” refers to any uncrosslinked hyaluronic acid polymer that may be used to form a hydrogel. Examples of hydrogel precursors include, but are not limited to, the amino-modified hyaluronic acid comprising a disulfide bond (HA-SS—NH₂).

In some embodiments, the hydrogel precursor includes a chemically-modified polymer. The chemically-modified HA polymer may form a three-dimensional network in an aqueous medium to form a hydrogel. In some embodiments, the chemically-modified HA polymer comprises a disulfide bond and terminal amine group.

In some embodiments, the primary amine of the functionalized hyaluronic acid can be reacted with a chemical crosslinker. In some embodiments, the hyaluronic acid polymer precursor (e.g., the uncrosslinked hyaluronic acid polymer) comprises a terminal amino group. In some embodiments, the primary amine group enables the functionalized hyaluronic acid to be chemically crosslinked. In some embodiments, the chemical crosslinker is polyethylene glycol (PEG) comprising a succinimidyl functional group (e.g., —NHS), as shown in FIGS. 1B and 1C. In some embodiments, the crosslinker comprises a buffer (e.g., phosphate buffer). In some embodiments, the chemical crosslinker is a multi-arm PEG. In some embodiments, the chemical crosslinker is a 3-arm PEG, a 4-arm PEG, a 6-arm PEG, an 8-arm PEG, or any combination thereof. In some embodiments, the chemical crosslinker is an 8-arm PEG.

In some embodiments, the crosslinker (e.g., PEG-NHS) is present at a concentration of about 1% w/v to about 30% w/v (e.g., about 1% to about 10%, about 1% to about 15%, about 1% to about 20%, about 1% to about 25%, about 1% to about 29%, about 5% to about 10%, about 5% to about 15%, about 5% to about 20%, about 5% to about 25%, about 5% to about 30%, about 10% to about 15%, about 10% to about 20%, about 10% to about 25%, about 10% to about 30% w/v, about 1% to about 50% w/v, about 1% to about 60% w/v, about 1% to about 70% w/v, about 50% to about 60% w/v, about 50% to about 70% w/v, about 5% to about 50% w/v, about 10% to about 50% w/v, about 20% to about 50% w/v, about 30% to about 50% w/v, or about 40% to about 50% w/v) in a buffer (e.g., phosphate buffer). In some embodiments, the crosslinker (e.g., PEG-NHS) is present at a concentration of about 10% w/v to in a buffer (e.g., phosphate buffer). In some embodiments, the crosslinker (e.g., PEG-NHS) is present at a concentration of about 50% w/v.

In some embodiments, the chemical crosslinker is PEG having a molecular weight ranging from about 5 kilodalton (kDa) to about 200 kDa (e.g., about 5 kDa to about 10 kDa, about 5 kDa to about 15 kDa, about 5 kDa to about 20 kDa, about 5 kDa to about 25 kDa, about 5 kDa to about 30 kDa, about 5 kDa to about 35 kDa, about 5 kDa to about 40 kDa, about 5 kDa to about 50 kDa, about 10 kDa to about 15 kDa, about 10 kDa to about 20 kDa, about 10 kDa to about 25 kDa, about 10 kDa to about 30 kDa, about 10 kDa to about 35 kDa, about 10 kDa to about 40 kDa, about 10 kDa to about 50 kDa, about 15 kDa to about 20 kDa, about 15 kDa to about 25 kDa, about 15 kDa to about 30 kDa, about 15 kDa to about 35 kDa, about 15 kDa to about 40 kDa, about 15 kDa to about 50 kDa, about 20 kDa to about 25 kDa, about 20 kDa to about 30 kDa, about 20 kDa to about 35 kDa, about 20 kDa to about 40 kDa, about 20 kDa to about 50 kDa, about 25 kDa to about 30 kDa, about 25 kDa to about 35 kDa, about 25 kDa to about 40 kDa, about 25 kDa to about 50 kDa, about 30 kDa to about 35 kDa, about 30 kDa to about 40 kDa, about 30 kDa to about 50 kDa, about 35 kDa to about 40 kDa, about 35 kDa to about 50 kDa, or about 40 kDa to about 50 kDa, about 5 kDa to about 100 kDa, about 5 kDa to about 150 kDa, about 5 kDa to about 200 kDa, about 50 kDa to about 100 kDa, about 50 kDa to about 150 kDa, about 50 kDa to about 200 kDa, about 100 kDa to about 150 kDa, about 100 kDa to about 200 kDa, or about 150 kDa to about 200 kDa). In some embodiments, the chemical crosslinker is PEG having a molecular weight of about 10 kDa. In some embodiments, the chemical crosslinker is PEG having a molecular weight of about 40 kDa.

In some embodiments, the chemical crosslinker comprises one or more PEG polymers having different molecular weights (e.g., any of the above-described molecular weights of PEG). For example, in some embodiments, the chemical crosslinker is a combination of a first PEG having a first molecular weight and a second PEG having a second molecular weight that is different than the first molecular weight. In some embodiments, the first PEG has a molecular weight of about 10 kDa, and the second PEG has a molecular weight of about 40 kDa. In some embodiments, the chemical crosslinker comprises a first PEG polymer and a second PEG polymer at a ratio of about 0:100 wt % to about 100:0 wt % (e.g., about 0:100 wt % to about 10:90 wt %, about 0:100 wt % to about 20:80 wt %, about 0:100 wt % to about 30:70 wt %, about 0:100 wt % to about 40:60 wt %, about 0:100 wt % to about 50:50 wt %, about 0:100 wt % to about 60:40 wt %, about 0:100 wt % to about 70:30 wt %, about 0:100 wt % to about 80:20 wt %, about 0:100 wt % to about 90:10 wt %, about 10:90 wt % to about 100:0 wt %, about 20:80 wt % to about 100:0 wt %, about 30:70 wt % to about 100:0 wt %, about 40:60 wt % to about 100:0 wt %, about 50:50 wt % to about 100:0 wt %, about 60:40 wt % to about 100:0 wt %, about 70:30 wt % to about 100:0 wt %, about 80:20 wt % to about 100:0 wt %, or about 90:10 wt % to about 100:0 wt %). In some embodiments, the chemical crosslinker comprises a first PEG polymer and a second PEG polymer at a ratio of about 70:30 wt %. In some embodiments, the chemical crosslinker comprises a first PEG polymer having a molecular weight of about 40 kDa and a second PEG polymer having a molecular weight of about 10 kDa at a ratio of about 70:30 wt %, respectively.

While the above-discussed hyaluronic acid polymer has been described and illustrated with respect to certain material formulations and methods of preparation, in some embodiments, a hyaluronic acid polymer that is otherwise substantially similar in formulation and function to the above-discussed hyaluronic acid polymer may include one or more materials formulations that are different from the ones discussed above or may be prepared using methods that are modified as compared to the methods described above. For example, while the hyaluronic acid polymer has been described and illustrated as including a terminal amino group, in some embodiments, a hyaluronic acid polymer that is otherwise substantially similar in formulation and function to the above-described hyaluronic acid polymer may alternatively include a thiol group instead of the amino group.

While above-discussed hyaluronic acid polymer has been described and illustrated as being crosslinked with a PEG crosslinker comprising a succinimidyl functional group, in some embodiments, a PEG crosslinker that is otherwise substantially similar in formulation and function to the above-discussed PEG crosslinker may include an ortho-pyridyl disulfide (OPSS) functional group instead of a succinimidyl functional group. For example, in some embodiments, the PEG crosslinker including the OPSS functional group crosslinks a chemically-modified HA polymer that is functionalized with a thiol group. In some embodiments, a PEG crosslinker that is otherwise substantially similar in formulation and function to the above-discussed PEG crosslinker may include an maleimide functional group instead of a succinimidyl functional group. For example, in some embodiments, the PEG crosslinker including the maleimide functional group crosslinks a chemically-modified HA polymer that is functionalized with a thiol group. In some embodiments, a PEG crosslinker that is otherwise substantially similar in formulation and function to the above-discussed PEG crosslinker may include an acrylate functional group instead of a succinimidyl functional group. For example, in some embodiments, the PEG crosslinker including the acrylate functional group crosslinks a chemically-modified HA polymer that is functionalized with a thiol group and/or an amine group. In some embodiments, the chemical crosslinker comprises one or more amine groups and a disulfide bond (e.g., NHS—SS—NHS).

### Physical Properties of MN Array Compositions

The physical properties of the microneedle array compositions of the disclosure include, but are not limited to, swelling ratio, mechanical strength, degradation rate, and drug release profile can be finely tuned by modulating the concentration, type, and/or molecular weight of the chemical crosslinker and/or the reducing agent. For example, in some embodiments, the molecular weight of the crosslinker can have a significant impact on the swelling ratio and mechanical strength of the amino-functionalized HA polymer comprising the disulfide bond. In some embodiments, the degradation rate is dependent on the concentration of the reducing agent that the amino-functionalized HA polymer is exposed to.

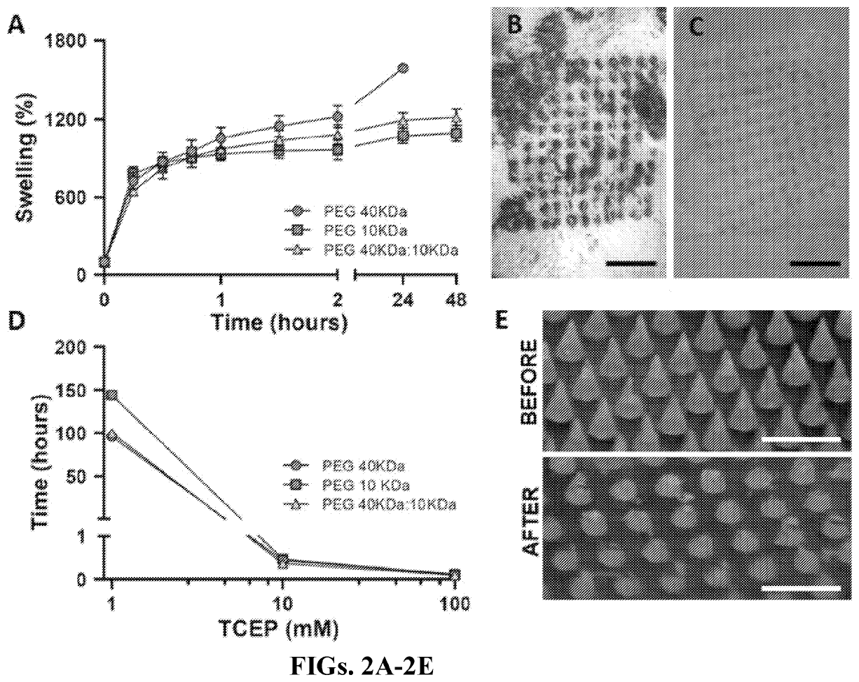

In some embodiments, the molecular weight of the crosslinker (e.g., PEG) is directly proportional to the swelling ratio of the hydrogel microneedles when measured after an extended period of time (e.g., 24 hours). In some embodiments, the molecular weight of the crosslinker does not have a significant impact on the swelling ratio of the hydrogel microneedles when measured after a short period of time (e.g., at about 2 hours at most). For example, as shown in FIG. 2A, crosslinking the HA polymer with a crosslinker (e.g., PEG) having a high molecular weight (e.g., 40 kDa) can lead to greater swelling ratio (e.g., about 1800%) after about 24 hours. In some embodiments, the microneedles have a swelling ratio ranging from about 600% to about 1300% (e.g., about 600% to about 700%, about 600% to about 800%, about 600% to about 900%, about 600% to about 1000%, about 600% to about 1100%, about 600% to about 1200%, about 600% to about 1250%, about 900% to about 1000%, about 900% to about 1100%, about 900% to about 1200%, or about 900% to about 1300%) after contacting the functionalized HA hydrogels with an aqueous environment for at least about 15 minutes to about 2 hours. In some embodiments, the microneedles have a swelling ratio ranging from about 1300% to about 1800% (e.g., about 1300% to about 1400%, about 1300% to about 1500%, about 1300% to about 1600%, about 1300% to about 1700%, about 1300% to about 1750%, about 1400% to about 1500%, about 1400% to about 1600%, about 1400% to about 1700%, about 1400% to about 1800%, about 1500% to about 1600%, about 1500% to about 1700%, about 1500% to about 1800%, about 1600% to about 1700%, about 1600% to about 1800%, or about 1700% to about 1800%) after contacting the functionalized HA hydrogels with an aqueous environment for at least about 2 hours to about 48 hours (e.g., about 2 hours to about 12 hours, about 2 hours to 24 hours, about 2 hours to 36 hours, about 2 hours to 48 hours, about 12 hours to 24 hours, about 12 hours to 36 hours, about 12 hours to 48 hours, about 24 hours to 36 hours, about 24 hours to 48 hours, or about 36 hours to 48 hours).

In some embodiments, the molecular weight of the crosslinker (e.g., PEG) is inversely proportional to the mechanical strength of the microneedles. For example, crosslinking the HA polymer with a crosslinker (e.g., PEG) having a low molecular weight (e.g., 10 kDa) can lead to greater mechanical strength that results in more efficient skin perforation and/or penetration as compared to a crosslinker (e.g., PEG) having a higher molecular weight (e.g., 40 kDa). In some embodiments, crosslinking the HA polymer with a combination of crosslinkers (e.g., PEG) having a low molecular weight (e.g., 10 kDa) and a higher molecular weight (e.g., 40 kDa) at specific ratios (e.g., 70% (wt %) 40 kDa and 30% (wt %) 10 kDa) can lead to greater mechanical strength that results in more efficient skin perforation and/or penetration as compared to a single crosslinker (e.g., PEG) having a higher molecular weight (e.g., 40 kDa).

In some embodiments, the degradation of the crosslinked HA polymer is controlled by the time and concentration of the reducing agent that the hydrogel is exposed to. In some embodiments, the crosslinked HA polymer hydrogels have on-demand degradation (e.g., the degradation can be rapidly dissolved or degraded within 30 seconds or less). In some embodiments, the concentration of the reducing agent is directly proportional to the degradation time. As shown in FIGS. 2D and 2E, the degradation of the hydrogels is almost instantaneous (e.g., within about 5 minutes or less) when exposed to about 100 mM of a reducing agent (e.g., TCEP). In some embodiments, the crosslinked HA polymer hydrogel is degraded within about 15 seconds (s) to about 10 minutes (min.) (e.g., about 15 s to about 1 min., about 15 s to about 2 min., about 15 s to about 3 min., about 15 s to about 4 min., about 15 s to about 5 min., about 15 s to about 6 min., about 15 s to about 7 min., about 15 s to about 8 min., about 15 s to about 9 min., about 15 s to about 10 min., about 30 s to about 1 min., about 30 s to about 2 min., about 30 s to about 3 min., about 30 s to about 4 min., about 30 s to about 5 min., about 30 s to about 6 min., about 30 s to about 7 min., about 30 s to about 8 min., about 30 s to about 9 min., about 30 s to about 10 min., about 1 min. to about 2 min., about 1 min. to about 3 min., about 1 min. to about 4 min., about 1 min. to about 5 min., or about 1 min. to about 10 min.) when exposed to about 75 mM to about 150 mM (e.g., about 75 mM to about 100 mM, about 80 mM to about 100 mM, about 90 mM to about 100 mM, about 100 mM to about 110 mM, about 100 mM to about 115 mM, about 100 mM to about 120 mM, about 100 mM to about 130 mM, about 100 mM to about 140 mM, or about 100 mM to about 150 mM) of a reducing agent (e.g., TCEP).

In some embodiments, the crosslinked HA polymer hydrogel is degraded within about 30 seconds (s) to about 20 minutes (min.) (e.g., about 30 s to about 1 min., about 30 s to about 2 min., about 30 s to about 3 min., about 30 s to about 4 min., about 30 s to about 5 min., about 30 s to about 6 min., about 30 s to about 7 min., about 30 s to about 8 min., about 30 s to about 9 min., about 30 s to about 10 min., about 1 min. to about 2 min., about 1 min. to about 3 min., about 1 min. to about 4 min., about 1 min. to about 5 min., or about 1 min. to about 10 min., about 30 s to about 11 min., about 30 s to about 12 min., about 30 s to about 13 min., about 30 s to about 14 min., about 30 s to about 15 min., about 30 s to about 16 min., about 30 s to about 17 min., about 30 s to about 18 min., about 30 s to about 19 min., about 30 s to about 19 min., about 1 min. to about 15 min., about 1 min. to about 20 min., about 5 min. to about 10 min., about 5 min. to about 15 min., about 5 min. to about 20 min., about 10 min. to about 15 min., about 10 min. to about 20 min., or about 15 min. to about 20 min.).

In some embodiments, the crosslinked HA polymer hydrogel is degraded when exposed to about 75 mM to about 150 mM (e.g., about 75 mM to about 100 mM, about 80 mM to about 100 mM, about 90 mM to about 100 mM, about 100 mM to about 110 mM, about 100 mM to about 115 mM, about 100 mM to about 120 mM, about 100 mM to about 130 mM, about 100 mM to about 140 mM, or about 100 mM to about 150 mM) of a reducing agent (e.g., TCEP).

In some embodiments, the crosslinked HA polymer hydrogel is degraded within about 10 min. to about 30 min. (e.g., about 10 min. to about 12 min., about 10 min. to about 15 min., about 10 min. to about 20 min., about 10 min. to about 25 min., about 10 min. to about 28 min., about 15 min. to about 20 min., about 15 min. to about 25 min., about 15 min. to about 30 min., about 20 min. to about 25 min., about 20 min. to about 30 min., or about 25 min. to about 30 min.) when exposed to about 5 mM to about 15 mM (e.g., about 5 mM to about 7.5 mM, about 5 mM to about 10 mM, about 5 mM to about 12.5 mM, about 5 mM to about 15 mM, about 10 mM to about 12.5 mM, or about 10 mM to about 15 mM) of a reducing agent (e.g., TCEP).

In some embodiments, the crosslinked HA polymer hydrogel is degraded within about 10 min. to about 30 min. (e.g., about 10 min. to about 12 min., about 10 min. to about 15 min., about 10 min. to about 20 min., about 10 min. to about 25 min., about 10 min. to about 28 min., about 15 min. to about 20 min., about 15 min. to about 25 min., about 15 min. to about 30 min., about 20 min. to about 25 min., about 20 min. to about 30 min., or about 25 min. to about 30 min.).

In some embodiments, the crosslinked HA polymer hydrogel is degraded when exposed to about 5 mM to about 15 mM (e.g., about 5 mM to about 7.5 mM, about 5 mM to about 10 mM, about 5 mM to about 12.5 mM, about 5 mM to about 15 mM, about 10 mM to about 12.5 mM, or about 10 mM to about 15 mM) of a reducing agent (e.g., TCEP).

In some embodiments, the crosslinked HA polymer hydrogel is degraded within about 50 hours (h) to about 150 h (e.g., about 50 h to about 75 h, about 50 h to about 100 h, about 50 h to about 125 h, about 50 h to about 150 h, about 75 h to about 100 h, about 75 h to about 125 h, about 75 h to about 150 h, about 100 h to about 125 h, about 100 h to about 150 h, about 125 h to about 150 h) when exposed to about 0.5 mM to about 2 mM (e.g., about 0.5 mM to about 1 mM, about 0.5 mM to about 1.25 mM, about 0.5 mM to about 1.5 mM, about 0.5 mM to about 1.75 mM, about 0.5 mM to about 1.9 mM, about 1 mM to about 1.25 mM, about 1 mM to about 1.5 mM, about 1 mM to about 1.75 mM, about 1 mM to about 2 mM, about 1.25 mM to about 1.5 mM, about 1.25 mM to about 1.75 mM, about 1.25 mM to about 2 mM, about 1.5 mM to about 1.75 mM, about 1.5 mM to about 2 mM, or about 1.75 mM to about 2 mM) of a reducing agent (e.g., TCEP).

In some embodiments, the crosslinked HA polymer hydrogel is degraded within about 50 hours (h) to about 150 h (e.g., about 50 h to about 75 h, about 50 h to about 100 h, about 50 h to about 125 h, about 50 h to about 150 h, about 75 h to about 100 h, about 75 h to about 125 h, about 75 h to about 150 h, about 100 h to about 125 h, about 100 h to about 150 h, about 125 h to about 150 h).

In some embodiments, the crosslinked HA polymer hydrogel is degraded when exposed to 0.5 mM to about 2 mM (e.g., about 0.5 mM to about 1 mM, about 0.5 mM to about 1.25 mM, about 0.5 mM to about 1.5 mM, about 0.5 mM to about 1.75 mM, about 0.5 mM to about 1.9 mM, about 1 mM to about 1.25 mM, about 1 mM to about 1.5 mM, about 1 mM to about 1.75 mM, about 1 mM to about 2 mM, about 1.25 mM to about 1.5 mM, about 1.25 mM to about 1.75 mM, about 1.25 mM to about 2 mM, about 1.5 mM to about 1.75 mM, about 1.5 mM to about 2 mM, or about 1.75 mM to about 2 mM) of a reducing agent (e.g., TCEP).

### Therapeutic Agents

In some embodiments, the HA hydrogel microneedles described herein include one or more therapeutic agents (e.g., as a drug delivery payload). In some embodiments, the one or more therapeutic agents are encapsulated within the polymeric, three-dimensional structure of each microneedle or other chemically modified HA polymeric structure or composition (e.g., a hydrogel disk or a hydrogel spray composition). In some embodiments, the therapeutic agents are encapsulated in, carried by, dispersed within, or otherwise loaded in or on the hydrogel microneedles or other chemically modified HA polymeric structure or composition (e.g., a hydrogel disk or a hydrogel spray composition). In some embodiments, one or more therapeutic agents are conjugated to a surface of the hydrogel microneedles or other chemically modified HA polymeric structure or composition (e.g., a hydrogel disk or a hydrogel spray composition). In some embodiments, a first therapeutic agent is encapsulated in, carried by, or otherwise loaded in or on a nanoparticle, a liposome, a micelle, a microparticle, an exosome, or the like. In some embodiments, a first therapeutic agent is encapsulated in, carried by, or otherwise loaded in or on a nanoparticle, a liposome, a micelle, a microparticle, an exosome, or the like, and a second therapeutic agent is outside the nanoparticle, liposome, micelle, microparticle, exosome, or the like within the same microneedle or other chemically modified HA polymeric structure or composition (e.g., a hydrogel disk or a hydrogel spray composition). In some embodiments, the first and second therapeutic agents are the same. In some embodiments, the first and second therapeutic agents are the different. In some embodiments, the therapeutic agents are dispersed, embedded, suspended, and/or mixed within the plurality of microneedles or other chemically modified HA polymeric structures.

In some embodiments, the plurality of microneedles or other chemically modified HA polymeric structure or composition (e.g., a hydrogel disk or a hydrogel spray composition) encapsulate a therapeutic agent at an encapsulation efficiency ranging from about 20% to about 95% (e.g., about 20% to about 25%, about 20% to about 30%, about 20% to about 40%, about 20% to about 50%, about 20% to about 55%, about 20% to about 60%, about 20% to about 65%, about 20% to about 70%, about 20% to about 75%, about 20% to about 80%, about 20% to about 85%, about 20% to about 90%, about 20% to about 95%, about 25% to about 30%, about 50% to about 55%, about 50% to about 60%, about 55% to about 60%, about 65% to about 70%, about 65% to about 75%, about 65% to about 80%, about 65% to about 85%, or about 65% to about 95%).

In some embodiments, the therapeutic agent is a hydrophobic therapeutic agent, a hydrophilic therapeutic agent, an amphiphilic therapeutic agent, or any combination thereof. In some embodiments, the therapeutic agent comprises an immunosuppressor. In some embodiments, the therapeutic agent comprises an immunoregulator. In some embodiments, the therapeutic agent comprises one or more of a chemokine, a chemotherapeutic, a nucleic acid, a protein (e.g., an antibody), a macromolecule, a nanoparticle, an exosome, a cytokine, a chemical-based drug (e.g., an anti-inflammatory therapeutic agent, a psychotropic drug, and/or anti-hypertensive drug), a growth factor, a vaccine, an immunosuppressor, an immunoregulator, or any combination thereof. In some embodiments, the therapeutic agent is a combination of two or more of any of the therapeutic agents listed herein. For example, in some embodiments, the HA hydrogel microneedles described herein comprise an anti-hypertensive drug (e.g., minoxidil) and one or more chemokines. In some embodiments, the HA hydrogel microneedles described herein comprise an anti-hypertensive drug (e.g., minoxidil) and one or more cytokines. In some embodiments, the HA hydrogel microneedles described herein comprise one or more chemokines and/or cytokines and one or more antibodies. In some embodiments, the HA hydrogel microneedles described herein comprise one or more chemokines and/or cytokines and one or more immunosuppressants (e.g., rapamycin).

In some embodiments, the immunosuppressor and/or the immunoregulator is a chemokine. In some embodiments, the chemokine comprises C-C motif chemokine 22 (CCL22) and/or interleukin-2 (IL-2). In some embodiments, the chemokine comprises chemokine (C-C motif) ligand 1 (CCL1), (C-C motif) ligand 2 (CCL2), (C-C motif) ligand 3 (CCL3), (C-C motif) ligand 4 (CCL4), (C-C motif) ligand 5 (CCL5), (C-C motif) ligand 6 (CCL6), (C-C motif) ligand 7 (CCL7), (C-C motif) ligand 8 (CCL8), (C-C motif) ligand 9 (CCL9), (C-C motif) ligand 10 (CCL10), (C-C motif) ligand 11 (CCL11), (C-C motif) ligand 12 (CCL12), (C-C motif) ligand 13 (CCL13), (C-C motif) ligand 14 (CCL14), (C-C motif) ligand 15 (CCL15), (C-C motif) ligand 16 (CCL16), (C-C motif) ligand 17 (CCL17), (C-C motif) ligand 18 (CCL18), (C-C motif) ligand 19 (CCL19), (C-C motif) ligand 20 (CCL20), (C-C motif) ligand 21 (CCL21), (C-C motif) ligand 22 (CCL22), (C-C motif) ligand 23 (CCL23), (C-C motif) ligand 24 (CCL24), (C-C motif) ligand 25 (CCL25), (C-C motif) ligand 26 (CCL26), (C-C motif) ligand 27 (CCL27), (C-C motif) ligand 28 (CCL28), chemokine (C-X-C motif) ligand 1 (CXCL1), chemokine (C-X-C motif) ligand 2 (CXCL2), chemokine (C-X-C motif) ligand 3 (CXCL3), chemokine (C-X-C motif) ligand 4 (CXCL4), chemokine (C-X-C motif) ligand 5 (CXCL5), chemokine (C-X-C motif) ligand 6 (CXCL6), chemokine (C-X-C motif) ligand 7 (CXCL7), chemokine (C-X-C motif) ligand 8 (CXCL8), chemokine (C-X-C motif) ligand 9 (CXCL9), chemokine (C-X-C motif) ligand 10 (CXCL10), chemokine (C-X-C motif) ligand 11 (CXCL11), chemokine (C-X-C motif) ligand 12 (CXCL12), chemokine (C-X-C motif) ligand 13 (CXCL13), chemokine (C-X-C motif) ligand 14 (CXCL14), chemokine (C-X-C motif) ligand 15 (CXCL15), chemokine (C-X-C motif) ligand 16 (CXCL16), chemokine (C-X-C motif) ligand 17 (CXCL17), chemokine (C motif) ligand 1 (XCL1), chemokine (C motif) ligand 2 (XCL2), chemokine (C-X3-C motif) ligand 1 (CX3CL1), or any combination thereof. In some embodiments, the therapeutic agents included in the HA hydrogel microneedles comprise one or more of any the chemokines described herein in combination with one or more of a cytokine, an antibody, an immunosuppressant (e.g., rapamycin), an anti-hypertensive (e.g., minoxidil), or any combinations thereof.

In some embodiments, the immunosuppressor and/or the immunoregulator is a chemokine receptor. In some embodiments, the therapeutic agent is a chemokine receptor. In some embodiments, the chemokine receptor comprises CC chemokine receptor 1 (CCR1), CC chemokine receptor 2 (CCR2), CC chemokine receptor 3 (CCR3), CC chemokine receptor 4 (CCR4), CC chemokine receptor 5 (CCR5), CC chemokine receptor 6 (CCR6), CC chemokine receptor 7 (CCR7), CC chemokine receptor 8 (CCR8), CC chemokine receptor 9 (CCR9), CC chemokine receptor 10 (CCR10), CC chemokine receptor 11 (CCR11), CXC chemokine receptor 1 (CXCR1), CXC chemokine receptor 2 (CXCR2), CXC chemokine receptor 3 (CXCR3), CXC chemokine receptor 4 (CXCR4), CXC chemokine receptor 5 (CXCR5), CXC chemokine receptor 6 (CXCR6), or any combination thereof. In some embodiments, the therapeutic agents included in the HA hydrogel microneedles comprise one or more of any the chemokine receptors described herein in combination with one or more of a cytokine, an antibody, an immunosuppressant (e.g., rapamycin), an anti-hypertensive (e.g., 10 minoxidil), or any combinations thereof.

In some embodiments, the immunosuppressor and/or the immunoregulator is a cytokine. In some embodiments, the cytokine comprises an interleukin (e.g., IL-2, IL-4, IL-7, IL-9, IL-13 and IL-15, IL-3, IL-6, IL-11, IL-13, IL-17A-F, IL-21, IL-22, IL-23, IL10, IL-35), transforming growth factor-beta (TGF-β), interferon-gamma (IFN-γ), tumor necrosis factor-alpha (TNF-α), or any combination thereof. In some embodiments, the therapeutic agents included in the HA hydrogel microneedles comprise one or more of any the cytokines described herein in combination with one or more of a chemokine, an antibody, an immunosuppressant (e.g., rapamycin), an anti-hypertensive (e.g., minoxidil), or any combinations thereof.

In some embodiments, the therapeutic agent comprises a nucleic acid. In some embodiments, the therapeutic agent can include a naturally occurring, modified, or synthetic nucleic acid such as, but not limited to, deoxyribonucleic acid (DNA), ribonucleic acid (RNA), viral vectors, chromosomes, aptamers, nucleosomes, or any combination thereof. Non-limiting examples of nucleic acids include DNA such as genomic DNA, methylated DNA, specific methylated DNA sequences, fragmented DNA, mitochondrial DNA, and RNA/DNA hybrids. Non-limiting examples of nucleic acids also include RNA such as various types of coding and non-coding RNA. Examples of the different types of RNA include messenger RNA (mRNA), ribosomal RNA (rRNA), transfer RNA (tRNA), microRNA (miRNA), viral RNA, CRISPR RNA (crRNA), trans-activating CRISPR RNA (tracrRNA), single guide RNA (sgRNA), and crRNA/tracrRNA hybrid. The RNA can be small (e.g., less than 200 nucleic acid bases in length) or large (e.g., RNA greater than 200 nucleic acid bases in length). Small RNAs mainly include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA). Further exemplary nucleic acids include, but are not limited to, recombinant nucleic acids, recombinant DNA, cDNA, genomic DNA, dsDNA, 5 RNA, siRNA, mRNA, saRNA, miRNA, lncRNA, tRNA, and shRNA. In some embodiments, the nucleic acid is homologous to a nucleic acid in a cell. In some embodiments, the nucleic acid is heterologous to a nucleic acid in a cell. In some embodiments, the nucleic acid is in the form of a plasmid. In some embodiments, the nucleic acid is a therapeutic nucleic acid. In some embodiments, the nucleic acid encodes a therapeutic polypeptide.

In some embodiments, the therapeutic agent comprises a protein. In some embodiments, the therapeutic agent comprises a polypeptide, or peptide, including, but not limited to, a structural protein, an enzyme, a cytokine (such as an interferon and/or an interleukin), a polyclonal or monoclonal antibody, or an effective part thereof, such as an Fv fragment, which antibody or part thereof, can be natural, synthetic or humanized, a peptide hormone, a receptor, or a signaling molecule. In some embodiments, the therapeutic agent comprises insulin. In some embodiments, the therapeutic agent comprises a hormone (e.g. a thyroid hormone such as levothyroxine or synthetic triiodothyronine). In some embodiments, the protein comprises an antibody (e.g., a single variable domain on a heavy chain (VHH) 20 antibody, a Nanobody®). In some embodiments, the antibody is anti-CD3 monoclonal Antibody.®). In some embodiments, the antibody is anti-CD3 monoclonal antibody. In some embodiments, the antibody comprises an anti-programmed death-1 (PD-1) monoclonal antibody, an anti-programmed death ligand-1 (PD-1) monoclonal antibody, an anti-vascular endothelial growth factor receptor (VEGFR) monoclonal antibody, an anti-cytotoxic T-lymphocyte-associated protein 4 (CTLA4) monoclonal antibody, or any combination thereof. In some embodiments, the antibody comprises an anti-CD3 monoclonal antibody, an anti-IL-6 monoclonal antibody, an anti-CD28 monoclonal antibody, an anti-CD52 monoclonal antibody, or any combination thereof. In some embodiments, the therapeutic agents included in the HA hydrogel microneedles comprise one or more of any the antibodies described herein in combination with one or more of a chemokine, a cytokine, an immunosuppressant (e.g., rapamycin), an anti-hypertensive (e.g., minoxidil), or any combinations thereof.

In some embodiments, the therapeutic agent comprises a macromolecule. In some embodiments, macromolecule comprises glucose. In some embodiments, the therapeutic agent includes, but is not limited to, at least one of a protein, a polypeptide, a peptide, a nucleic acid, a virus, a virus-like particle, an amino acid, an amino acid analogue, a modified amino acid, a modified amino acid analogue, a steroid, a proteoglycan, a lipid and a carbohydrate or a combination thereof (e.g., chromosomal material comprising both protein and DNA components or a pair or set of effectors, wherein one or more convert another to active form, for example catalytically).

In some embodiments, the therapeutic agent comprises a nanoparticle. Non-limiting examples of nanomaterials or nanoparticles that can be delivered using the methods and compositions of the disclosure include quantum dots, plasmonic nanoparticles, metallic nanoparticles, polymeric nanoparticles, liposomes, lipid nanoparticles, exosomes, or any combination thereof. In some embodiments, the nanomaterial or nanoparticle has a size (e.g. a diameter) of less than about 300 nm. In some embodiments, the nanomaterial or nanoparticle can have a diameter of between about 2 nm to about 200 nm (e.g., between about 10 nm to about 30 nm, between about 5 nm to about 25 nm, between about 10 nm to about 25 nm, between about 15 nm to about 25 nm, between about 20 nm and about 25 nm, between about 25 nm to about 50 nm, between about 50 nm and about 200 nm, between about 70 nm and about 200 nm, between about 80 nm and about 200 nm, between about 100 nm and about 200 nm, between about 140 nm to about 200 nm, and between about 150 nm to about 200 nm).

In some embodiments, the nanomaterial or nanoparticle can be spherical or ellipsoidal, or can have an amorphous shape. In some embodiments, the nanomaterial or nanoparticle can be magnetic (e.g., include a core of a magnetic material). In some embodiments, the magnetic material or particle can contain a diamagnetic, paramagnetic, superparamagnetic, or ferromagnetic material that is responsive to a magnetic field.

In some embodiments, the nanomaterial or nanoparticle can contain, in part, a core and/or a shell of containing a polymer (e.g., poly(lactic-co-glycolic acid)). Skilled practitioners will appreciate that any number of art known materials can be used to prepare nanoparticles, including, but are not limited to, gums (e.g., Acacia, Guar), chitosan, gelatin, sodium alginate, and albumin. Additional polymers that can be used to generate the nanomaterial or nanoparticle to be dispersed in the cell solution are known in the art. For example, polymers that can be used to generate the nanomaterial or nanoparticle include, but are not limited to, cellulosics, poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactides (PLA), polyglycolides (PGA), poly(lactide-co-glycolides) (PLGA), polyanhydrides, polyorthoesters, polycyanoacrylate and polycaprolactone.

In some embodiments, the chemical-based drug comprises an anti-inflammatory therapeutic agent. In some embodiments, the therapeutic agent is a corticosteroid. Exemplary chemical-based drug for inclusion in the compositions include, but are not limited to, an antibacterial agent, an anti-fungal agent, an anti-viral agent, an anti-acanthamoebal agent, an immunosuppressive agent, an anti-vascular endothelial growth factor (anti-VEGF) agent, a growth factor, or any combination thereof.

In some embodiments, the therapeutic agent is a psychotropic drug (e.g., an anti-depressant or an anti-epileptic drug). Non-limiting examples of anti-depressants include a tricyclic antidepressant (e.g. secondary amine tricyclic antidepressant or a tertiary amine tricyclic antidepressant), a selective serotonin reuptake inhibitor, a serotonin and noradrenaline reuptake inhibitor, a reversible monoamine oxidase inhibitor and a monoamine oxidase inhibitor, nortriptyline, desipramine, amitriptyline, imipramine, fluoxetine, citalopram, paroxetine, fluvoxamine, escitalopram (lexapro), sertraline, venlafaxine, moclobemide, phenelzine, duloxetine, and tranylcypromine. Non-limiting examples of anti-epileptic drugs includes carbamazepine, valproate, ethosuximide and phenyloin. In some embodiments, the psychotropic drug is arapiprazole, olanzapine, quetiapine, risperidone, or ziprasidone.

In some embodiments, the therapeutic agent is an anti-inflammatory therapeutic agent. Non-limiting examples of suitable anti-inflammatory agents include a steroidal anti-inflammatory drug (e.g., prednisolone), a non-steroidal anti-inflammatory drug (e.g., bromfenac), an mTOR inhibitor (e.g., rapamycin), a calcineurin inhibitor, a synthetic or natural anti-inflammatory protein, methylprednisolone, prednisolone, hydrocortisone, fludrocortisone, prednisone, celecoxib, ketorolac, piroxicam, diclorofenac, ibuprofen, and ketoprofen, rapamycin, cyclosporin, and tacrolimus/FK-506. In some embodiments, the immunosuppressant is rapamycin.

In some embodiments, the therapeutic agent is an anti-hypertensive drug. Non-limiting examples of anti-hypertensive drugs include a vasodilator (e.g., minoxidil), an angiotensin-converting enzyme (ACE) inhibitors (e.g., captopril, benazepril, enalapril, enalaprilat, fosinopril, lisinopril, quinapril, ramipril, and trandolapril or others with similar molecular mechanisms), a calcium channel blocker (e.g., nifedipine, verapamil, nicardipine, diltiazem, isradipine, amlodipine, nimodipine, felodipine, nisoldipine, bepridil or others with similar molecular mechanisms), a beta-blocker (e.g., atenolol, metoprolol, propranolol, timolol, nadolol, acebutolol, pindolol, sotalol, labetalol, oxprenolol or others with similar molecular mechanisms), methyldopa, hydralazine hydrochloride, labetalol, adenosine, nifedipine, and magnesium sulfate. In some embodiments, the therapeutic agents included in the HA hydrogel microneedles comprise any of the anti-hypertensive drugs listed herein (e.g., minoxidil) and one or more chemokines and/or cytokines. In some embodiments, the therapeutic agents included in the HA hydrogel microneedles comprise one or more of any the anti-hypertensive drugs described herein in combination with one or more of a chemokine, a cytokine, an antibody, an immunosuppressant (e.g., rapamycin), or any combinations thereof.

In some embodiments, the therapeutic agent is a growth factor. In some embodiments, the growth factor includes, but is not limited to, epithelial growth factor, fibroblast growth factor, nerve growth factor, hepatocyte growth factor, or any combination thereof. Further non-limiting examples of suitable growth factors include transforming growth factors (TGFs) (e.g., beta transforming growth factors such as, TGF-β1, TGF-β2, TGF-β3), fibroblast growth factors (FGFs), platelet derived growth factors (PDGFs), epidermal growth factors (EGFs), connective tissue activated peptides (CTAPs), osteogenic factors, bone morphogenetic proteins (e.g., BMP-1, BMP-2, BMP-3, BMP-4, BMP-5, BMP-6, BMP-7, BMP-8, BMP-9); heparin-binding growth factors (e.g., fibroblast growth factor (FGF), epidermal growth factor (EGF), insulin-like growth factor (IGF)), Inhibins (e.g., Inhibin A, Inhibin B), growth differentiating factors (for example, GDF-1), and Activins (e.g., Activin A, Activin B, Activin AB), and biologically active analogs, fragments, and derivatives of such growth factors.

In some embodiments, the therapeutic agent is a vaccine. In some embodiments, the therapeutic agent is an mRNA vaccine. In some embodiments, the therapeutic agent is a vaccine comprise an RNA that encodes highly immunogenic antigens capable of eliciting potent neutralizing antibodies responses against coronavirus antigens, such as Severe Acute Respiratory Syndrome (SARS)-CoV-2 coronavirus antigens. In some embodiments, the therapeutic agent is a “booster” vaccine. As used herein, when referring to a prophylactic composition, such as a vaccine, the term “booster” refers to an extra administration of the prophylactic (vaccine) composition. A booster (or booster vaccine) may be given after an earlier administration of the prophylactic composition.

In some embodiments, the hydrogel composition further includes a pharmaceutically acceptable carrier. As used herein, the expression “pharmaceutically acceptable carrier” refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation and is compatible with administration to a subject, for example a human. It must also be suitable for use in contact with any tissues or organs with which it may come in contact, meaning that it must not carry a risk of toxicity, irritation, allergic response, immunogenicity, or any other complication that excessively outweighs its therapeutic benefits. Examples of pharmaceutically acceptable carriers include, but are not limited to, a solvent or dispersing medium containing, for example, water, pH buffered solutions (e.g., phosphate buffered saline (PBS), HEPES, TES, MOPS, etc.), isotonic saline, Ringer's solution, polyol (for example, glycerol, propylene glycol, liquid polyethylene glycol, and the like), alginic acid, ethyl alcohol, and suitable mixtures thereof. In some embodiments, the pharmaceutically acceptable carrier can be a pH buffered solution (e.g. PBS).

In some embodiments, the pharmaceutically acceptable carrier is a topical carrier. In some embodiments, the composition is formulated for topical use. In some embodiments, the composition is topically administered to a tissue (e.g., a skin tissue) of a patient. In some embodiments, the composition can be applied to a tissue (e.g., a skin tissue) for topical, targeted delivery of a therapeutic agent.

Methods of Treatment and/or Sampling

The present disclosure features methods of treating a disease (e.g., a skin disorder or an immune disorder) in a subject in need thereof using the chemically-modified hyaluronic acid compositions described herein (e.g., microneedle arrays composed of the above-described hyaluronic acid polymer comprising a disulfide bond and a terminal amine group). The hydrogel compositions (e.g., hydrogel microneedle array compositions) of the disclosure may be used for the prevention and/or treatment of a wide variety of skin disorders and immune disorders (e.g., autoimmune diseases).

As used herein, “prevention” includes reducing a risk of developing a disease. As used herein, “treatment,” refers to inhibition of progression of a disease (e.g., a skin disorder or an immune disorder), stasis of symptoms, partial or full amelioration of symptoms, partial or full eradication of the skin condition, disease, or disorder, and partial or full eradication of the immune condition, disease, or disorder. Thus, treatment includes partial or total alleviation of symptoms and/or reduction of signs or symptoms of illness.

The present disclosure features methods of treating a skin disorder in a subject in need thereof using the chemically-modified hyaluronic acid microneedle array compositions described herein (e.g., the microneedle arrays composed of the above-described hyaluronic acid polymer comprising a disulfide bond and a terminal amine group). The present disclosure also features methods of locally suppressing an immune response in a tissue of a subject using the chemically-modified hyaluronic acid microneedle array compositions described herein.

In some embodiments, the methods include contacting any of the microneedle array compositions described herein with a skin surface of the subject. Next, in some embodiments, the methods further include applying pressure on the microneedle array such that the penetrating tip of each microneedle of the plurality of microneedles penetrates the skin surface. In some embodiments, the tip portion of the microneedles perforates and/or penetrates the stratum corneum. In some embodiments, the microneedles penetrate the epidermis and/or the dermis. In some embodiments, the microneedles create a pathway or pore within a skin tissue (e.g., within the stratum corneum, epidermis, and/or dermis) and release the therapeutic agent beneath the skin surface. In some embodiments, once released, the therapeutic agent can directly go into the systemic circulation without facing the barrier of the stratum corneum. In some embodiments, once released, the therapeutic agent can remain within a localized area of the tissue near the microneedle injection site. In some embodiments, releasing the therapeutic agent beneath the skin surface does not elicit a systemic response. In some embodiments, the methods include maintaining the microneedle array in place for about 5 minutes to about 1 week (e.g., about 5 min. to about 10 min., about 5 min. to about 15 min., about 5 min. to about 20 min., about 5 min. to about 30 min., about 5 min. to about 1 hour (h), about 5 min. to about 2 h, about 5 min. to about 3 h, about 5 min. to about 6 h, about 5 min. to about 12 h, about 5 min. to about 18 h, about 5 min. to about 24 h, about 5 min. to about 36 h, about 5 min. to about 48 h, about 5 min. to about 72 h, about 5 min. to 4 days, about 5 min. to 5 days, about 5 min. to 6 days, about 5 min. to 7 days, about 10 min. to about 15 min., about 10 min. to about 20 min., about 10 min. to about 30 min., about 10 min. to about 1 hour (h), about 10 min. to about 2 h, about 1 h to about 2 h, about 1 h to about 3 h, about 1 h to about 6 h, about 1 h to about 12 h, about 1 h to about 24 h, about 1 day to about 2 days, about 1 day to about 2 days, about 1 day to about 3 days, about 1 day to about 4 days, about 1 day to about 5 days, about 1 day to about 6 days, about 1 day to about 7 days, or more) after the penetrating tip of each microneedle of the plurality of microneedles penetrates the skin surface.

The present disclosure features methods include of sampling an interstitial fluid of a subject in need thereof. In some embodiments, the methods include simultaneous delivery of a therapeutic agent and sampling of an interstitial fluid. In some embodiments, the methods include delivery of a therapeutic agent without sampling of the interstitial fluid. In some embodiments, the methods sampling of the interstitial fluid without simultaneous delivery of a therapeutic agent. In some embodiments, the methods include steps similar as those described above for delivery of a therapeutic agent. For example, in some embodiments, the methods include contacting the microneedle array of the disclosure with a skin surface of the subject. Next, in some embodiments, the methods further includes applying pressure on the microneedle array such that the penetrating tip of each microneedle of the plurality of microneedles penetrates the skin surface, thereby absorbing the interstitial fluid.

In some embodiments, the hydrogel microneedles can absorb the skin interstitial fluid once they penetrate through the stratum corneum, epidermis, and/or dermis. In some embodiments, the methods further include removing the microneedle array from the skin surface after the interstitial fluid is absorbed. In some embodiments, the methods include removing the microneedle array from the skin surface after a period of time ranging from about 5 min. to about 24 hours (e.g., about 5 min. to about 10 min., about 5 min. to about 15 min., about 5 min. to about 30 min., about 5 min. to about 1 h, about 5 min. to about 2 h, about 5 min. to about 3 h, about 5 min. to about 6 h, about 5 min. to about 12 h, about 5 min. to about 18 h, about 5 min. to about 24 h, about 30 min. to about 1 h, about 1 h to about 2 h, about 1 h to about 4 h, about 1 h to about 6 h, about 1 h to about 12 h, about 1 h to about 24 h, about 6 h to about 12 h, about 6 h to about 18 h, about 6 h to about 24 h, about 12 h to about 18 h, or about 12 h to about 24 h). Next, in some embodiments, the methods include degrading the plurality of microneedles after removing the microneedle array from the skin surface. In some embodiments, the plurality of microneedles is degraded by contacting the plurality of microneedles with a reducing agent, thereby extracting the interstitial fluid from the plurality of microneedles. In some embodiments, contacting the plurality of microneedles with a reducing agent does not cause cell death and does not negatively affect (e.g., degrade, contaminate, or otherwise interfere with the stability of) one or more components of the interstitial fluid (e.g., a cell, biomarker, antibody, or any combination thereof). In some embodiments, contacting the plurality of microneedles with a reducing agent does not impedes the phenotyping of one or more components of the interstitial fluid (e.g., a cell, biomarker, antibody, or any combination thereof) (e.g., there is no cleavage/degradation of cell receptors). In some embodiments, the degrading of the plurality of microneedles is facilitated by the cleavage of the disulfide bond present in the degradable HA polymer. In other words, in some embodiments, the disulfide bond is configured to be cleaved upon exposure to the reducing agent. In some embodiments, the reducing agent is TCEP. In some embodiments, the reducing agent is glutathione, dithiothreitol, or beta-mercaptoethanol. In some embodiments, the plurality of microneedles is contacted with the reducing agent at any of the reducing agent concentrations disclosed elsewhere herein.

In some embodiments, the interstitial fluid comprises a biomarker and/or a cell. In some embodiments, the biomarker is a cytokine or a chemokine (e.g., any of the cytokines and/or chemokines disclosed elsewhere herein). In some embodiments, the biomarker is glucose. In some embodiments, the biomarker is caffeine. In some embodiments, the biomarker is an orally- and/or systemically-administered drug (e.g., nonsteroidal anti-inflammatory drug such as ibuprofen, naproxen, or the like). In some embodiments, the hydrogel microneedles can be coupled with a wearable device for in situ detection of biomarkers using electrochemical and optical techniques. For example, in some embodiments, the microneedle array can extract a skin interstitial fluid comprising glucose, and the methods disclosed herein include determining a level of glucose in the ISF that can be indicative of a blood glucose level of a patient. In some embodiments, the wearable device can be a continuous glucose monitor that is operatively connected with the microneedle array.

Non-limiting examples of one or more biomarkers that can be detected in situ using the microneedle array described herein include, but are not limited to, bilirubin, carnosine, cortisol, creatine, creatinine, homocysteine, uric acid, vitamin A, vitamin B12, lumazine, pantothenic acid (vitamin B5), lumichrome, pyridoxamine (vitamin B6 form), pyridoxal (vitamin B6 form), 4-pyridoxic acid, ascorbic acid (vitamin C), ergocalciferol (vitamin D2), 7-dehydrocholesterol (vitamin D3), hypoxanthine, nicotinamide adenine dinucleotide (NAD), uridine, xanthine, myristic acid (c14:0), palmitoleic acid (c16:1), stearic acid (c18:0), arachidic acid (c20:0), cholic acid, glycocholic acid, urocanic acid, 4-Guanidinobutanoic acid, succinylhomoserine, tocopherol, N6-(delta2-isopentenyl)-adenine, nebularine, cytidine monophosphate (CMP), cytidine, inosine, 3-methyladenine, nicotinamide ribotide, N-methyltryptamine, sphingosine, 20-COOH-leukotriene B4, stachyose, gulonolactone, fructose 6-phosphate, rhamnose, oxalic acid, phosphoenolpyruvic acid, diethanolamine, cyclohexane-1,2-diol, triethanolamine, methyl jasmonate, or any combinations thereof. In some embodiments, the interstitial fluid biomarker that can be sampled with the methods and microneedle arrays described herein comprises a sugar, salt, fatty acid, amino acid, co-enzyme, enzyme, hormone, neurotransmitter, or any combination thereof.

In some embodiments, one or more cells found in the interstitial fluid can be sampled with the methods and microneedle arrays described herein comprises. In some embodiments, the one or more cells that can be sampled include, but are not limited to, T-cell, B-cell, a natural killer (NK) cell, a neutrophil, a macrophage, a monocyte, a dendritic cell, a memory T cell, a regulator T cell, a myeloid-derived suppressor cell (MDSC), or any combination thereof. In some embodiments, the microneedles of the disclosure can release a therapeutic agent that attracts a specific type of cell and further recruit and extract that specific type of cell. In some embodiments, the cell recruited and extracted by the microneedle array compositions of the disclosure is an immune cell. In some embodiments, the immune cell is a lymphoid progenitor (e.g., a T cell, B cell, Natural Killer cell, or the like). In some embodiments, the immune cell is a myeloid progenitor (e.g., a dendritic cell, a macrophage, or the like). In some embodiments, the immune cell is a T-cell, B-cell, a natural killer (NK) cell, a neutrophil, a macrophage, a monocyte, a dendritic cell, a memory T cell, a regulator T cell, a myeloid-derived suppressor cell (MDSC), or any combination thereof. In some embodiments, the cell is a T cell (e.g., a regulatory T cell). In some embodiments, the therapeutic agent used to recruit the cell is a cytokine, a chemokine, an antibody, or any combination thereof (e.g., any of the cytokines, chemokines, and/or antibodies disclosed elsewhere herein). In some embodiments, the microneedles of the disclosure can release a therapeutic agent that repels and/or depletes a specific type of cell within a target tissue site. For example, in some embodiments, the microneedles of the disclosure can release an anti-CD3 monoclonal antibody for T cell (e.g., CD3+-T cell) depletion therapy.

In some embodiments, the methods further comprise inducing an immunoregulation of a tissue (e.g., a skin tissue) of a subject in need thereof. In the event of a challenge such as burn, autoimmune disease, and/or foreign organ transplant, the skin immune network can detect danger signals and resolve inflammation. CD4⁺CD25⁺FoxP3⁺ regulatory T cells (T_{reg}) are a subtype of T cells that suppress other activated immune cells and control body's response to self and foreign antigens, in order to prevent overactivated immune responses such as in the case of autoimmune disorders. T_{regs} account for one of the largest subsets of immune cells in the skin, promoting local immunological homeostasis and restoring normal function after a threat. The increased proportion of T_{regs} in skin-resident CD4⁺ T cell population compared to other organs (20% in skin vs. 5% in peripheral blood) also suggest an integral role for immune regulation in a tissue-specific manner. Indeed, disruption in skin T_{regs} homeostasis, due to dysregulated T_{regs} number or function, triggers disorders such as psoriasis, alopecia areata, alopecia totalis, alopecia universalis, androgenetic alopecia, diffuse systemic scleroderma, atopic dermatitis, or cutaneous lupus erythematosus.

Skin allograft transplantation (from a genetically different individual) is the first-line therapy for severe burn patients and victims of traumatic injuries when autograft transplantation (from self) is not viable, due to insufficient healthy tissue for excision, donor site morbidity, or poor tolerability to additional surgeries. However, skin rejection following skin allotransplantation, which is the most immunogenic of all known allografts, is inevitable, arising from the intense immunogenicity of transplanted allografts harboring immunogenic antigens presented to the recipient's immune system. The current gold-standard therapy for the management of skin rejection is systemic immunosuppression, which partially suppresses rejection, at the cost of increasing the risk of opportunistic infections and incidence of malignancy. Since T_{regs} mediate specific functions depending entirely upon their residing tissues, tissue-specific therapeutic approaches should be favored to maximize their efficacy.

Hence, in some embodiments, the methods of the disclosure include inducing substantial recruitment and localized expansion of T_{regs} as way to manage and/or treat autoimmune disorders and skin transplantation. For example, in some embodiments, the microneedle array compositions can delivery chemokines and/or cytokines (e.g., CCL22 and IL-2) to induce substantial recruitment and localized expansion of T_{regs} in a tissue (e.g., a skin allograft), thereby tilting the effector-to-T_{regs} cell ratio at the site of allo-immunity in favor of immunological homeostasis. In some embodiments, the methods described herein include recruiting endogenous and adoptively transferred T_{regs} to restore the tolerogenic environment at a localized tissue (e.g., a skin transplant site). In some embodiments, the methods include using the microneedle array compositions to locally delivery a plurality of T_{regs} to a tissue (e.g. a skin tissue) of a subject in need thereof.

In some embodiments, the methods of treatment include using the microneedle array compositions to locally deliver IL-10, TGF-β, IL33, IFN-alpha, IFN-β, indoleamine 2,3-dioxygenase (IDO), programmed death 1 (PD1), fatty acid synthase (FAS), Fas receptor, CTLA4, human leukocyte antigen G (HLA-G), anti-CD3 monoclonal antibody, thymoglobulin, anti-CD52 monoclonal antibody, cyclophosphamide, Janus kinase (JAK) inhibitors, rapamycin, CTLA4-Ig, azathioprine, cyclophosphamide, methotrexate, cyclosporine, cortico-steroids, or any combination thereof.

In some embodiments, the methods include decreasing an inflammatory response and/or preventing an increase of an inflammatory response in a tissue (e.g., a skin tissue) of a subject in need thereof. In some embodiments, the methods include treating and/or preventing an infection in a tissue (e.g., a skin tissue) of a subject in need thereof. In some embodiments, the methods include treating a wound in a tissue (e.g., a skin tissue) of a subject in need thereof. In some embodiments, the methods include promoting and/or enhancing wound healing in a tissue (e.g., a skin tissue) of a subject in need thereof. As used herein, the terms “promoting and/or enhancing wound healing” refer to either the induction of the formation of granulation tissue of wound contraction and/or the induction of epithelialization (e.g., the generation of new cells in the epithelium). In some embodiments, the methods include locally delivering an anti-inflammatory agent and/or an anti-bacterial agent alone or in combination with a chemokine and/or a cytokine (e.g., CCL22 and IL-2) or a cell (e.g., a plurality of T_{regs}) to a tissue (e.g., a skin tissue).

### Skin and Immune Disorders

Non-limiting skin and immune disorders that can be treated using the hydrogel compositions of the disclosure include, but are not limited to, burns (e.g., first degree burns, second degree burns, and/or third degree burns), topic dermatitis, contact dermatitis, drug-induced delayed type cutaneous allergic reactions, toxic epidermal necrolysis, cutaneous T-cell lymphoma, bullous pemphigoid, alopecia areata, alopecia totalis, alopecia universalis, androgenetic alopecia, vitiligo, acne rosacea, prurigo nodularis, scleroderma, herpes simplex viral skin infections, acne, rosacea, eczema, keloids, psoriasis, pruritus, scleroderma, post-inflammatory hyperpigmentation, melasma, skin cancers, or any combination thereof. In some embodiments, the skin disorders that can be treated using the hydrogel compositions of the disclosure include, but are not limited to, diffuse systemic scleroderma, atopic dermatitis, cutaneous lupus erythematosus, alopecia areata, alopecia totalis, alopecia universalis, androgenetic alopecia, vitiligo, psoriasis, or any combination thereof. In some embodiments, the skin disorders that can be treated using the hydrogel compositions of the disclosure include, but are not limited to, Behcet's disease, dermatitis herpetiformis, dermatomyositis, lichen planus, linear IgA disease, lupus of the skin, morphea, scleroderma, ocular cicatrical pemphigoid, pemphigoid, pemphigus, vasculitis, psoriasis, and psoriatic arthritis.

Non-limiting indications that can be treated using the hydrogel compositions of the disclosure include immune responses associated with skin transplantation (e.g., allografts) and/or autoimmune disorders. In some embodiments, the methods include locally inducing an immunoregulation in a burned tissue, an autograft, a split-thickness skin graft, a full-thickness skin graft, an allograft, a homograft, a xenograft, a meshed graft, a sheet graft, or any combination thereof. In some embodiments, the methods include locally suppressing an immune response in a burned tissue, an autograft, a split-thickness skin graft, a full-thickness skin graft, an allograft, a homograft, a xenograft, a meshed graft, a sheet graft, or any combination thereof.

### EXAMPLES

Certain embodiments of the present disclosure are further described in the following examples, which do not limit the scope of any embodiments described in the claims.

### Example 1—Fabrication of Hyaluronic Acid (HA)-Based Microneedle (MNs), In Vitro Studies, and In Vivo Studies

### Materials

All reagents and solvents were purchased from Sigma Aldrich unless otherwise stated. Sodium hyaluronate (60 kDa) was obtained from LifeCore Medical with a purity of at least 95%. NHS-terminated 8-arm PEG was purchased from Creative PEG Works. MN PDMS custom-made molds (11×11 array of needles having a height of 600 m, a base width of 300 μm, and tip-to-tip spacing of 600 m) were obtained from Blueacre Technology. CCL22 and IL-2 chemokines were purchased from PeproTech.

### Synthesis of Amino-Modified Hyaluronic Acid (HA-SS—NH₂) Polymer

60 kDa-sodium hyaluronate (1% w/v in MES buffer) was activated with N-(3-(dimethylamino)propyl)carbodiimide (EDC) and N-hydroxysuccinimide (NHS) at a 1:4:2 molar ratio and reacted at room temperature for 30 minutes. The activated hyaluronic acid (HA) was then mixed with cysteamine dihydrochloride at a 1:10 molar ratio and reacted at room temperature for 12 hours. HA-SS—NH₂ was purified by dialysis against deionized water for 6 days at room temperature, freeze dried, and stored at −20° C. protected from humidity until use. For structural analysis, modified HA-SS—NH₂ was dissolved in D20 and analyzed by ¹H-NMR, recorded using a 400 MHz Varian NMR spectrometer (NMR Instruments, Clarendon Hills, IL).

### Fluorescent Labelling of HA-SS—NH₂ Polymer

HA-SS—NH₂ polymer was fluorescently tagged with AlexaFluor® 647 carboxylic acid succinimidyl ester (AF647) at a 1:0.1 molar ratio (HA-SS—NH₂:AF647). HA-SS—NH₂ polymer in 0.1 M bicarbonate buffer (pH 8.5) was mixed with AF647 and reacted for 1 hour (h) at room temperature in the dark. HA-SS—NH₂-AF647 polymer was washed with PBS and recovered by centrifugal filtration (10 kDa MWCO, Millipore) at 14000 revolutions per minute (RPM) for 15 min at 4° C.

### Ha Hydrogel Disks Fabrication

100 μL of hydrogel disks for streamlined screening were prepared by mixing equal volumes of HA-SS—NH₂ polymer (10% w/v) and the 8-arm-PEG-NHS crosslinker (10% w/v). Solutions were dissolved separately with phosphate buffer (pH=7.4) and vigorously mixed together for 10 seconds inside cylindrical plastic molds (diameter: 5.00 mm; height: 2.50 mm). Hydrogel disks were allowed to react for 5 minutes to ensure full gelation, freeze-dried, and stored at room temperature protected from humidity until use.

### Ha-Based Microneedle (MN) Fabrication

MNs were produced using custom-made molds consisting in a 11×11 array of negative MNs projections, each one with a height of 600 μm and a radius of 150 μm. First, HA-SS—NH₂ polymer (10% w/v in phosphate buffer, pH=7.4) was casted on top of the molds and centrifuged at 4200 rpm for 5 minutes. Excess polymer was removed, and molds were freeze-dried. Then, 8-arm-PEG-NHS crosslinker (10% w/v in phosphate buffer, pH=7.4) was casted and forced by centrifugation through the mold under the same conditions. This approach, together with the gradual gelation of hydrogel, ensured a successful polymerization of the matrix from “tip-to-top” of the MNs and a homogenous composition. Excess polymer was carefully removed, and the molds were freeze-dried. Next, an aqueous solution containing chemokines and glycine (10 μg mL⁻¹) was deposited and briefly spun for 15 seconds. Immediately after, a polymeric backing layer of PLGA (Resomer® RG 858 S, Sigma-Aldrich, USA) at 15% (w/v) dissolved in acetonitrile was added dropwise until covering the whole area of the mold. Finally, HA-based MNs were allowed to dry at room temperature for 12 hours, peeled off the molds carefully, and stored at room temperature preserved from humidity.

Swelling Studies with HA-Based Hydrogel Disks

HA-based hydrogels disks were incubated with PBS at 37° C. and their weight was recorded over time (Wᵢ) and normalized to their respective dry weight (W₀). Swelling percentage was assessed as a function of mass increase over time and calculated as: Wᵢ×100/W₀.

### Skin Penetration Studies

Penetration capacity of the HA-based MNs was tested ex vivo and in vivo in shaved C57BL/6 mice. HA-based MNs were applied and kept in place using medical-grade tape (FLEXcon, USA) for 1 hour. Skin penetration was confirmed by surface staining with blue Shandon™ Tissue-Marking Dye (Thermo Fisher) and further imaged by optical microscopy.

### On-Demand Digestion of HA-Derived Hydrogel Matrices

HA-based MNs or HA-based hydrogels were incubated with 10 mM Tris (2-carboxyethyl) phosphine (TCEP) solution in supplemented cell culture media or PBS at pH 7.4 (depending on whether cells were collected). HA-based MNs or HA-based hydrogels were incubated under rotation at 37° C. for 10 minutes and the recovered suspension was filtered with a 70 μm cell strainer (BD Biosciences) to remove any impurities.

### Cell Lines

Human monocyte THP-1 cells (ATTC) were maintained in RPMI 1640 supplemented with 10% FBS, 2 mM L-glutamine, 25 mM HEPES, 100 μg/mL Normocin™ (InvivoGen) and 100 U/mL penicillin and 100 μg/mL streptomycin. Cell lines were maintained in a humidified incubator at 37° C., 5% CO₂.

### Cytotoxicity Studies

THP-1 cells were incubated with different concentrations of digestion media (TCEP solution in supplemented media), ranging from 0.1 mM to 100 mM, for 10 minutes. Thereafter, digestion media was removed, and cells were stained using the LIVE/DEAD™ fixable Violet Dead Cell Stain Kit (Thermo Fisher Scientific, USA) following the manufacturer's guidelines. Dead cells were analyzed by flow cytometry using a BD LSRFortessa™ flow cytometer.

### Study of Chemokine Release Kinetics

Recombinant human IL-2 was fluorescently labeled with the Lightning-Link® Rapid Alexa Fluor®594 kit (Novus Biologicals, USA) following the manufacturer's instructions. Release studies were conducted with HA-based MNs loaded with labeled IL-2. IL-2-loaded, HA-based MNs were placed in Eppendorf Tubes®, immersed with phosphate buffered saline (PBS) (1 mL), and incubated under rotation at 37° C. 100 μL of PBS was replaced at predetermined time point, and IL-2 fluorescence was assessed by checking the fluorescence at 650-665 nm using a microplate reader.

### Ha-Based MNs Loading Capacity

HA-based MNs were loaded with labeled IL-2 concentrations. IL-2-loaded MNs were digested as described above, and IL-2 loading capacity was assessed by checking the IL-2 fluorescence at 650-665 nm using a microplate reader.

Recovery of Immune Cells from HA-Based MNs

HA-based MN patches were incubated with 1×10⁶ THP-1 cells per well for 24 hours. Then, MNs were washed with PBS to minimize unspecific interactions between cells and the MN backing layer and digested as described above. After digestion, cells were pelleted and stained with the CellTrace™ CFSE Cell Proliferation Kit Protocol (Thermo Fisher Scientific, USA) following the manufacturer's instructions. Total number of infiltrated cells was quantified via flow cytometry using a BD® LSR II flow cytometer.

### Recovery of Soluble Analytes in a Mimetic Skin Model

Analyte recovery capacity of hydrogel-based MNs was conducted using a skin model attempting to mimic the mechanical properties of the epidermis/ISF interface. 1.4% w/v agarose hydrogels containing increasing amounts of the model metabolite Rhodamine B were polymerized in 30 mm×15 mm petri dishes and covered and with a stretched layer of parafilm aiming to emulate the properties of the water-impermeable stratum corneum. MN patches were left inserted in the agarose gels for two hours to reach a swelling plateau and subsequently digested as previously described. Finally, absorbance of the recovered analytes was measured in a plate reader (λ=553 nm) and correlated with the extracted mass.

### Animal Experiments

C57BL/6J (B6 wild type; #000664), BALB/cJ (BALB/c wild type; #000651) and B6.129S7-Rag1tm1Mom (B6 Rag1−/−, #002216) mice were purchased from The Jackson Laboratory (Bar Harbour, ME, USA) and housed under specific-pathogen-free conditions at the Brigham and Women's Hospital animal facility. All mouse work was performed in compliance with ethical regulations and was approved by the Institutional Animal Care and Use Committee of the Brigham and Women's Hospital. Age and sex-matched mice (6-8 weeks, male and female) were randomized into experimental and control groups for all experiments.

### Murine Skin Transplantation

A fully MHC-mismatched murine skin transplant model was used. Briefly, full-thickness trunk skin grafts (1.0 cm by 1.5 cm) from BALB/c donors were harvested and connective, adipose, and panniculus carnosus tissues were cleared using blunt-tipped forceps. The fur of each anesthetized recipient Rag1−/− mouse was shaved at the dorsal trunk, 1.0 cm by 1.5 cm of the recipient mouse's skin was excised, and an equally-sized skin graft was sutured onto the graft bed with 6-0 PROLENE® polypropylene sutures (Ethicon, #8695G). Skin transplants were secured with dry gauze and bandaged for 7 days before adoptive cell transfer and MN application

### Analysis of Immune Infiltrate by Flow Cytometry

A single cell suspension of skin grafts was obtained. In brief, skin grafts were harvested, minced into 0.5 mm fragments and digested in a solution of RPMI supplemented with 10% Fetal Bovine Serum, 1% Penicillin and Streptomycin (100 IU/ml Penn, 100 μg/ml Strep) and collagenase P (stock concentration 1 mg/ml; cat no.: 11213865001, Roche) for 3 h at 37° C. Afterwards, skin grafts were re-incubated for 15 minutes at room temperature after adding 200 Kunitz units/ml of recombinant DNase I (cat no.: 10104159001, Roche) to reduce DNA fragments and clumping. The solution was then filtered through 70 M mesh filters and centrifuged at 800 units of gravity (g) for 8 minutes, then resuspended in full media supplemented with 20% FBS and incubated at 37° C. overnight to recover from enzymatic treatment. Then cells were resuspended in fluorescence-activated cell sorting (FACS) staining buffer (1× Dulbecco's phosphate-buffered saline (DPBS), 1.0% bovine serum albumin, 0.02% sodium azide (Sigma-Aldrich)) for flow cytometry analysis. Cells were counted manually using a hemocytometer and stained with fluorescent antibodies at a maximum concentration of 1×10⁶ cells in 100 μL FACS staining buffer (BioLegend). The following anti-mouse antibodies were purchased from eBioscience™: CD45 PE-Cy7 (clone 30-F11), Foxp3 APC (clone FJK-16S), CD4 Pacific Blue™ (clone RM4-5), CD4 PE (clone RM4-5) and BD® Biosciences: CD3 PerCP Cy5.5 (clone 500A2), CD8 Alexa Fluor® 488 (clone 53-6.7). Dead cells were stained using Fixed Viability Dye eFluor™ 780 (eBioscience™). Stained cells were analyzed by flow cytometry using a BD FACSCanto™ II cytometer (BD® Biosciences) and all data were analyzed using FlowJo version 10 (FlowJo, LLC).

### CCL22-Dependent Trans-Well Migration Assay

Recruitment of T_{regs} as a function of CCL22 was assessed in a 24-well plate containing a 5 μm-pore polycarbonate Transwell® filter system (Costar Transwell® permeable support #3421). MNs loaded with incremental amounts of CCL22 (0.10 and 100 ng) or equivalent dose of soluble CCL22 were added in the receiver wells and incubated at 37° C. for 1 hour. 5×10⁵ CD3⁺ T cells were magnetically isolated from C57BL/6 mice using mouse T cell isolation kit (EasySep™, #19851) and resuspended in full culture media and added to the top wells. Cells were then incubated at 37° C. and 5% CO₂ for 3 hours. Cells in the receiver well were then harvested and stained with Fixed viability dye, anti-CD3, CD4, CD25, and Foxp3 antibodies and analyzed by flow cytometry.

### Quantitative Real-Time Polymerase Chain Reaction

Quantitative real-time polymerase chain reaction (qPCR) was used to assess the differential expression of mRNA transcripts between control (Empty MN) and CCL22+IL-2 (10 ng or 100 ng) groups. A small piece of each allograft was kept in RNAprotect® tissue reagent (QIAGEN, Cat. No. #76104). On the same day, skin allografts were harvested for digestion and flow cytometry analysis. Later, the skin grafts were partially thawed and 1 mm² pieces were cut to finer pieces. RNA was then isolated using RNA isolation kit (QIAGEN RNeasy® plus Mini Kit, Cat. #4136) following the manufacturer's protocol. Eluted RNA concentration was measured. The RNA concentration was measured with a NanoDrop™ 2000 Spectrophotometer (Thermo Fisher Scientific, Waltham, MA, United States) and complementary DNA strands were reverse transcribed with iScript™ Reverse Transcription Supermix (#1708841, Bio-Rad Laboratories) as per the manufacturer's protocol, and the product was stored at −20° C. until further use. Quantitative real-time PCR was performed in 0.1 mL MicroAmp™ Fast Optical 96-Well Reaction Plates (Applied Biosystems, #4346906) with 30 ng of ds-RNA per mRNA target, 500 nM forward and 500 nM reverse primers, and SsoAdvanced™ Universal SYBR® Green Supermix (#1725274, Bio-Rad Laboratories) diluted to 1× with PCR-grade water (#W4502, Sigma-Aldrich) in 10-μL reaction volumes.

Primer pairs were based on OnGene's qSTAR qPCR Primer Pairs (Rockville, MD, United States) and synthesized through Integrated DNA Technologies (Coralville, IA, United States). Cycle threshold (Ct) values were measured with QuantStudio™ 3 (Thermo Fisher Scientific, Waltham, MA, United States). Ct values were then corrected with GAPDH housekeeping gene expression per replicate, per run, log 2 normalized, averaged for the control replicates, and deviation from the average was calculated per condition, per replicate. Fold change of Foxp3 to CD3 was calculated by dividing fold change of Foxp3 to fold change of CD3 (each separately normalized to GAPDH) and fold change of IL-6 was presented after normalization to GAPDH. The primers sequences used for qPCR analysis are shown in Table S2 below.

TABLE S2
Primers sequences used for qPCR analysis.

| Primer | Forward Sequence | Reverse Sequence |
|---|---|---|
| mGAPDH | 5′-CATCACTGCCACCCAGAAGACTG-3′ | 5′-ATGCCAGTGAGCTTCCCGTTCAG-3′ |
| | (SEQ ID NO: 1) | (SEQ ID NO: 2) |
| mFOXP3 | 5′-CCTGGTTGTGAGAAGGTCTTCG-3′ | 5′-TGCTCCAGAGACTGCACCACTT-3′ |
| | (SEQ ID NO: 3) | (SEQ ID NO: 4) |
| mCD3 | 5′-GCTCCAGGATTTCTCGGAAGTC-3′ | 5′-ATGGCTACTGCTGTCAGGTCCA-3′ |
| | (SEQ ID NO: 5) | (SEQ ID NO: 6) |
| mIL-6 | 5′-TACCACTTCACAAGTCGGAGGC-3′ | 5′-CTGCAAGTGCATCATCGTTGTTC-3′ |
| | (SEQ ID NO: 7) | (SEQ ID NO: 8) |

### Statistical Analysis

Statistical analyses were carried out using Graph-Pad Prism 8 (GraphPad Software). All data are reported as mean+standard error of the mean (SEM). For in vitro experiments, a minimum of n=3 biological replicates were used per condition in each experiment. Pairwise comparisons were performed using Student t-tests. Multiple comparisons among groups were determined using one-way ANOVA followed by a post-hoc test. For in vivo experiments, a minimum of n=4 biological replicates were used per condition in each experiment. Multiple comparisons among groups were determined using non-parametric t test (Mann-Whitney). No specific pre-processing of data was performed prior to statistical analyses. Differences between groups were considered significant at p-values below 0.05 (* p<0.05, ** p<0.01, *** p<0.001).

### Example 2—Design of a Highly Swellable Microneedle Platform with On-Demand Degradation

In this study, hyaluronic acid (HA)-based MNs were developed to deliver immunomodulators while enabling simultaneous sampling of the cellular fraction in the skin interstitial fluid (ISF). HA is a biocompatible, non-immunogenic, and FDA-approved linear polysaccharide. HA natural ability to absorb a large volume of water makes it an ideal candidate for rapid ISF extraction. In addition, HA can function as a natural ligand of the ubiquitous CD44 receptor, providing a binding motif for the cells present in ISF. Here, the HA polymer backbone was chemically modified to allow the formation of a digestible HA hydrogel capable of extracting both cellular and soluble biomarkers that are present in ISF upon patch retrieval. HA was modified with cysteamine dihydrochloride, harboring both a primary amine group (for hydrogel formation) and a disulfide bond (for hydrogel degradation) (FIG. 1B). To form the HA-based hydrogel, HA primary amines were reacted with the 8-arm-PEG-NHS crosslinker containing a succinimidyl functional group (FIG. 1B), allowing for spontaneous hydrogel formation without the use of external triggers. For MN fabrication (top scheme of FIG. 2B), the HA-derived polymer was cast into a mold, centrifuged at high speed and freeze-dried, creating a porous matrix that provide the scaffold to which subsequent components would be added. Next, a PEG crosslinker was added, followed by the addition of an aqueous solution containing the immunomodulators IL-2 and CCL22. Finally, a backing-layer was deposited, serving as an anchor for MN administration and retrieval. The disulfide bond in the modified HA backbone allowed for on-demand cleavage to release the migrated cells following ISF sampling upon the addition of a reducing agent, tris (2-carboxyethyl) phosphine (TCEP), a water-soluble and non-toxic reducing agent amenable for biochemical applications). The addition of TCEP facilitated the collapse of the 25 tri-dimensional structure of the hydrogel MNs and in turn, the release of the entrapped cells (FIG. 1C).

### Example 3—Biophysical Characterization of the HA-Based MNs

To assess the HA-based MNs performance, the MNs swelling capacity, mechanical strength, and on-demand digestion were studied. The influence of the crosslinking agent in determining the swelling capacity of the HA-NH₂-derived hydrogels as a function of weight gain over time was first investigated. Three different crosslinking agents were screened, differing in their molecular weight: (1) 40 kDa-8-arm-PEG-NHS (40 kDa-PEG), (2) 10 kDa-8-arm-PEG-NHS (10 kDa-PEG) and (3) their combination 70:30 wt % of 40 kDa:10 kDa-PEG. Here, hydrogel disks (rather than MNs) that provided with about 30-fold increase in volume were chosen to be used to effectively screen for the best crosslinking agent. It was found that the crosslinker type did not affect the initial swelling phase; an average swelling ratio of about 800% in less than an hour was observed for all hydrogels (FIG. 2A). However, the crosslinker type had a marked influence on swelling at later time points with ratios of 1073%±58% and 1592%±36% for hydrogels derived from the 10 kDa-PEG and 40 kDa-PEG, respectively. As expected, when the mixture of both crosslinkers was used the swelling ratio was in between these values; 1191%±59%. The results show that the choice of crosslinker, specifically, its molecular weight, allowed mediating the swelling capacity of the HA-derived hydrogels without the need of adding osmolytes such as sucrose or maltose to increase the osmotic pressure. Similar swelling phenomena were observed in stability studies where hydrogels were incubated in physiologically-relevant conditions. The PEG-40 kDa-derived-hydrogels dissolved after 48 hours of incubation while the volume of the other hydrogels remained unchanged for extended periods of time. Next, the capacity of the MN patch to penetrate the skin was evaluated both ex vivo and in vivo using a mouse model. Fine-tuning the crosslinking agent, both in terms of molecular weight and relative ratio, served as a powerful strategy to improve the mechanical strength of the MNs, avoiding the use of solid core/shells and irreversible crosslinking strategies. The ability to form the entire needles from the hydrogels provides with higher volume for entrapment of ISF biomarkers, and thus, permits better immune cell sampling. The ex vivo studies revealed that MNs containing 10 kDa-PEG (alone or when mixed with the 40 kDa-PEG) could efficiently disrupt the stratum corneum as confirmed by the accumulation of blue Tissue-marking dye inside the micro conduits (FIG. 2B), while those that were solely crosslinked with the PEG-40 kDa could not penetrate the viable skin. Similar results were observed in vivo (FIG. 2C). Thus, the 70:30 40 kDa:10 kDa-8-arm-PEG-NHS was chosen for the remaining studies given that this formulation provided both a high swelling capacity and adequate mechanical properties that enabled it to pierce the mouse skin.

The ability to induce on-demand digestion of the MNs using TCEP was also characterized. It was found that TCEP was highly biocompatible and did not affect cell viability. Study of the digestion kinetics of the HA-derived hydrogels confirmed a linear correlation between TCEP concentration and the time needed for full hydrogel digestion (FIG. 2D). High concentrations of TCEP (>10 mM) ensured complete digestion in half an hour whereas hydrogel digestion was not accomplished, or was far too long, when TCEP concentration was lower than 1 mM. Considering these findings, the working concentration for further studies was chosen to be 10 mM TCEP. The digestion kinetics of these hydrogels when in a MN form was then studied. It was confirmed that complete digestion of the MN array occurred in less than 5 minutes (FIG. 2E). The MNs high surface-to-volume ratio and low overall volume (˜3 μL) (compared to the hydrogel disks) facilitated the penetration of the reducing agent and accelerated their digestion.

### Example 4—HA-Based MNs for Simultaneous Drug Delivery and ISF Sampling

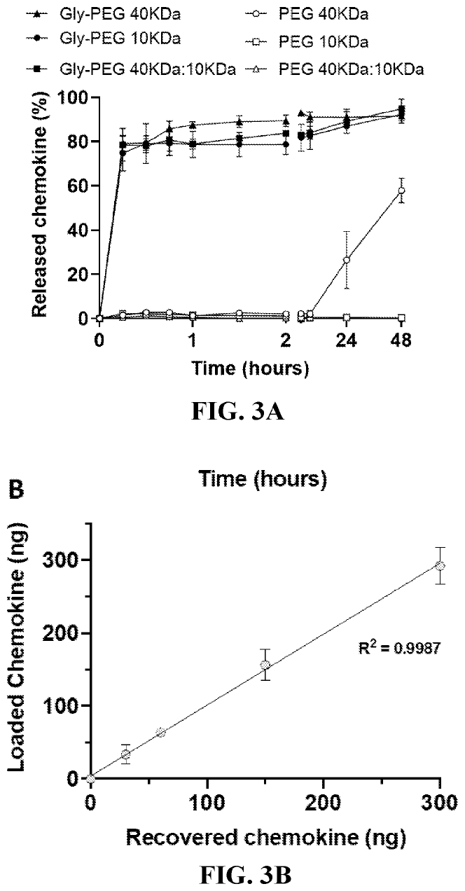

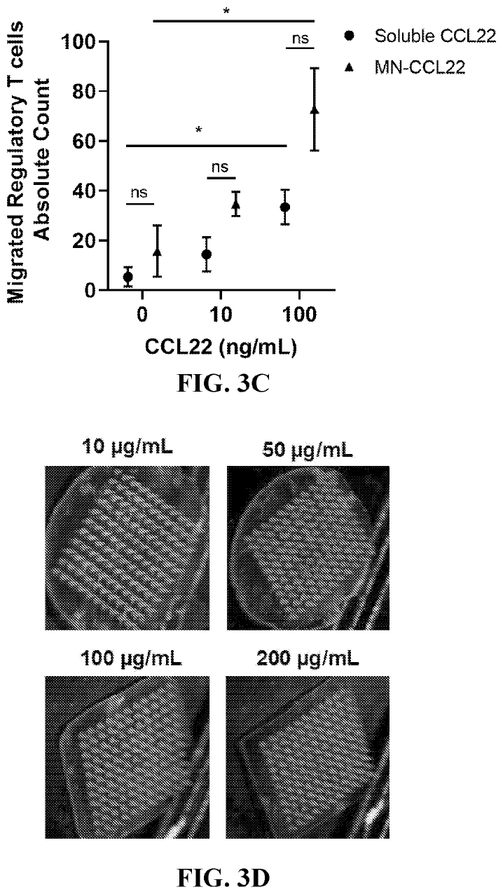

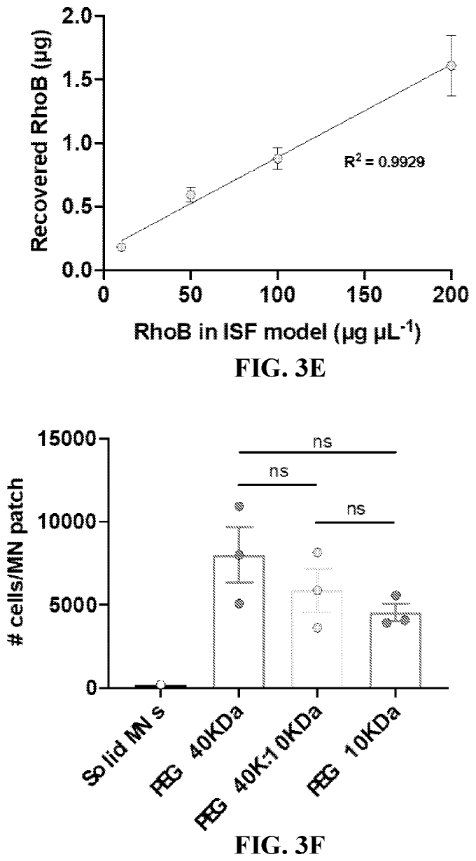

The efficient release delivery of cytokines from the MNs was initially studied using fluorescently labeled IL-2. Interestingly, no IL-2 was released (FIG. 3A), which was attributed to the interaction between the IL-2-amine groups with unreacted free-NHS groups in the crosslinker, impeding its release. To validate this hypothesis, the interaction with the cytokine was blocked by treating the hydrogel with an end-capping agent, the amino acid glycine, whose amino-end groups would preferentially interact with the crosslinker's non-reacted NHS-terminal groups. Indeed, end-capping the hydrogel resulted in more than 80% of the cytokine being released within the first 30 minutes, making it the chosen strategy for further studies. Next, the loading efficiency of IL-2 was studied by fluorescence quantification, revealing a linear correlation between the loaded concentration and the retrieved ones after digestion of the MN patches (FIG. 3B), thereby confirming the accuracy of the loading method and the potential to deliver biologically relevant dosages in the nanogram range.

Next, the biological activity of chemokines when loaded in the MNs was examined. Specifically, the ability of CCL22 to mediate T_{regs} recruitment when delivered using the MN platform through a trans-well migration assay was studied. Briefly, ranging concentrations of CCL22, either MN-doped or soluble, were incubated with CD3⁺ T cells, and the number of migrated T_{regs} was analyzed after 3 hours of incubation by flow cytometry. The results indicated a dose-dependent increase of T_{regs} migration for both soluble and MN-loaded CCL22 wells (P=0.02 and 0.04 for soluble and MN respectively). Recruitment of T_{regs} by CCL22-containing MNs was comparable to that following the addition of soluble CCL22 at the same dose (p=0.9, 0.2, 0.2 for 0, 10 and 100 ng/ml), suggesting that the chemokine functionality was not affected by the fabrication method or by being stored at room temperature prior to the experiments (for about 1 week). A trend of a higher T_{regs} recruitment in wells with CCL22-loaded MNs was seen compared to soluble CCL22. This can stem from the maintenance of a higher gradient of CCL22 when released from the MNs compared to soluble CCL22 (FIG. 3C).

In parallel, the diagnostic capacity of the platform and its ability to extract ISF in the absence of chemotactic agents was studied. Sampling of soluble biomarkers using MNs was investigated using a mimetic skin model. Briefly, agarose gels mimicking the mechanical properties of the epidermis/ISF were covered with a stretched layer of parafilm that emulated the water-impermeable stratum comeum. Hydrogel-based MNs were applied to the skin model containing a model analyte, Rhodamine B (RhoB), which was recovered after digestion of the patches. Differences in RhoB concentration could be easily detected by gross observation as evidenced by the color change of the MN matrix after administration (FIG. 3D). Quantification of the analyte absorbance confirmed a linear correlation between the concentration of RhoB in the skin-mimetic hydrogel and the concentration of the retrieved ISF when sampled using the PEG-40 kDa:10 kDa-derived MNs (FIG. 3E).

Extraction of the cellular component of ISF was assessed by incubating the arrays of MNs in monocyte-like cell suspensions followed by their digestion to collect and measure the infiltrated cellular fraction. Arrays of solid MNs were also incorporated to the analysis as controls to discern whether recovered cells were embedded within the hydrogel matrix or originated from unspecific interactions with the MN walls. Quantification of the digested suspension by flow cytometry depicted that cells were diffusing into the hydrogel MNs and remained inside the matrix (FIG. 3F). As expected, the number of cells recovered from the solid arrays (i.e., “solid MNs”) was practically negligible.

### Example 5—Delivery of CCL22 and IL-2 to Skin Allografts Via MNs Results in Increased T_{Regs} Recruitment and Reduced Inflammation In Vivo

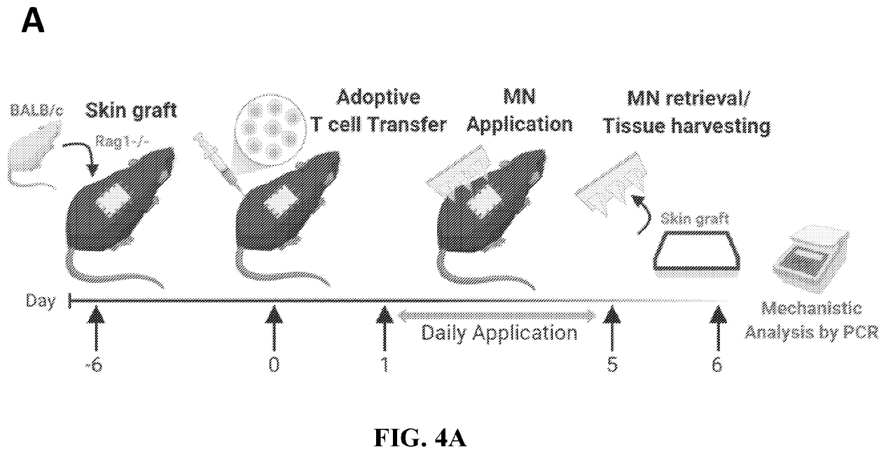

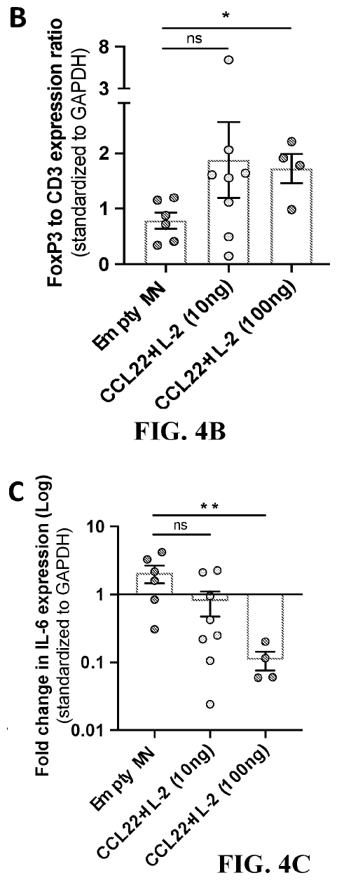

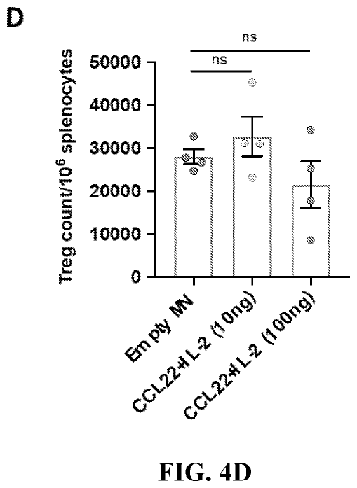

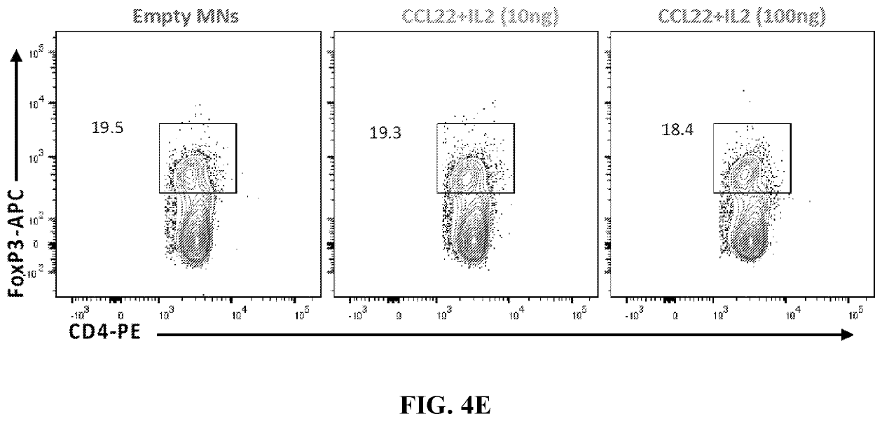

After exploring the ability of the MNs to rapidly release chemokines in vitro, the MN local immune modulatory function in vivo was studied using a fully mismatched major histocompatibility complex (MHC) skin transplant model, which is a highly immunogenic model of rejection. MNs loaded with CCL22 and IL-2 were synthesized, at two different concentrations (100 ng CCL22+10 ng IL-2 or 100 ng CCL22+100 ng IL-2), with the rationale being for CCL22 to enhance the recruitment of T_{regs}, and for IL-2 to maintain T_{regs} homeostasis in vivo. A 10 mm×15 mm skin patch recovered from a BALB/cJ mouse was then grafted onto the dorsal trunk of a immunodeficient recombination activation gene 1 knockout (Rag^{-/-}) mouse on C57BL/6 background, which lack both T and B cells. Skin graft was dressed and left to heal for 5 days. On day 6 post-transplant, 7×10⁶ magnetically isolated T lymphocytes were adoptively transferred to induce rejection in the allograft recipients. One day post-adoptive transfer, MNs (100 ng CCL22+10 ng IL-2 or 100 ng CCL22+100 ng IL-2) were applied daily on the skin allograft for five consecutive days, with one group of mice receiving empty MNs as a control. Recipient mice were euthanized at day 7 post T_{regs} adoptive transfer and skin graft harvested and studied by quantitative PCR to study tissue cell infiltration and gene expression (FIG. 4A). Indentations on the mouse skin were observed, which indicated successful penetration of the MNs to the epidermis layer, and without causing any major injury or bleeding.

Using quantitative PCR technique, the differential expression of CD3 as universal T cell marker (cell infiltrates inducing allograft rejection), FoxP3 as a transcription factor differentiating T_{regs} from conventional T cells (T_{regs} suppress rejection), and IL-6 as a major pro-inflammatory cytokine involved in promoting graft rejection, normalized to the expression of the house keeping gene GAPDH was studied. Ratio of fold change in FoxP3 to CD3 expression was then used as an indicator of the effect of the delivered therapy on T_{regs} recruitment and proliferation compared to no treatment (FIG. 4B). Skin allografts treated with CCL22+100 ng IL-2 showed significantly increased ratio of FoxP3 to CD3 compared to allografts treated with empty MNs (p=0.03). Although skin grafts treated with CCL22 and lower IL-2 dose of 10 ng showed a trend of higher FoxP3 to CD3 ratio compared to controls, the difference was not statistically significant (p=0.1). This suggests a dose-dependent effect of IL-2 on T_{regs} proliferation. Treatment of the allografts with the combination of CCL22 and IL2 resulted in reduced expression of IL-6, with grafts treated with CCL22+100 ng IL-2 showing more than ten times reduction in IL-6, indicating reduced inflammation at the 30 allograft site (p=0.009) (FIG. 4C).

### Example 6—Delivery of CCL22 and IL-2 with MNs Did not Induce T_{Regs} Expansion in Peripheral Organs

To study the systemic effects of MN-mediated local delivery of IL-2, T_{regs} populations were evaluated in splenocytes harvested from allograft recipients on day 7 post-adoptive transfer. The data showed comparable T_{regs} numbers for all the groups and no significant T_{regs} expansion in the spleens (FIGS. 4D and 4E). It has been shown that systemic administration of IL-2 promoted T_{regs} proliferation in the spleen but failed to do so in the skin allografts which aggravated their outcomes. Here, it was confirmed that minimal systemic effects occurred following MN-based delivery of IL-2, suggesting the enhanced safety of the platform when compared to systemic routes and the potential to modulate immune cell composition and reduce inflammatory state locally.

### Example 7—Cells Captured by MNs can be Continuously Monitored Following Retrieval and Rapid MN Digestion

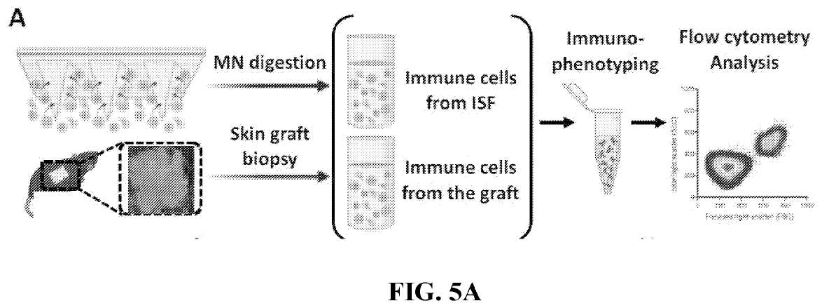

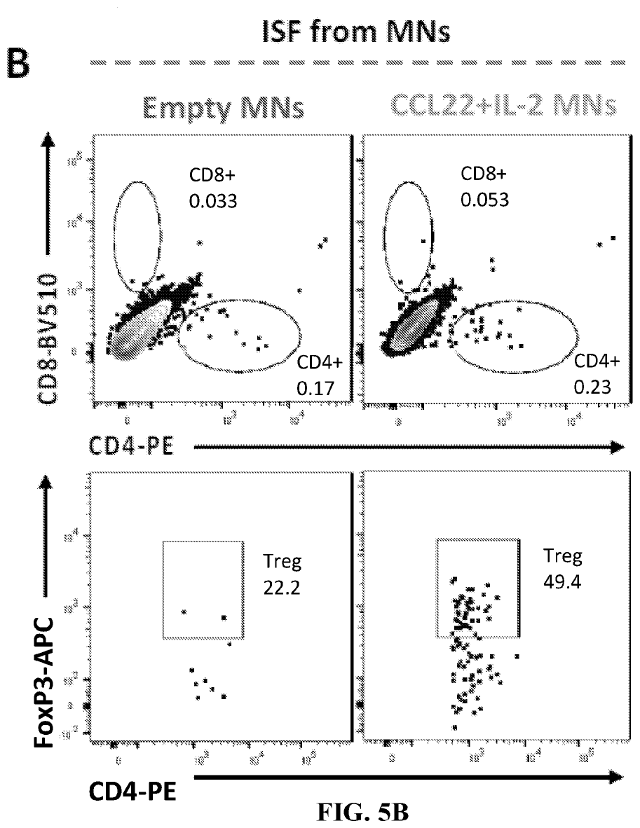

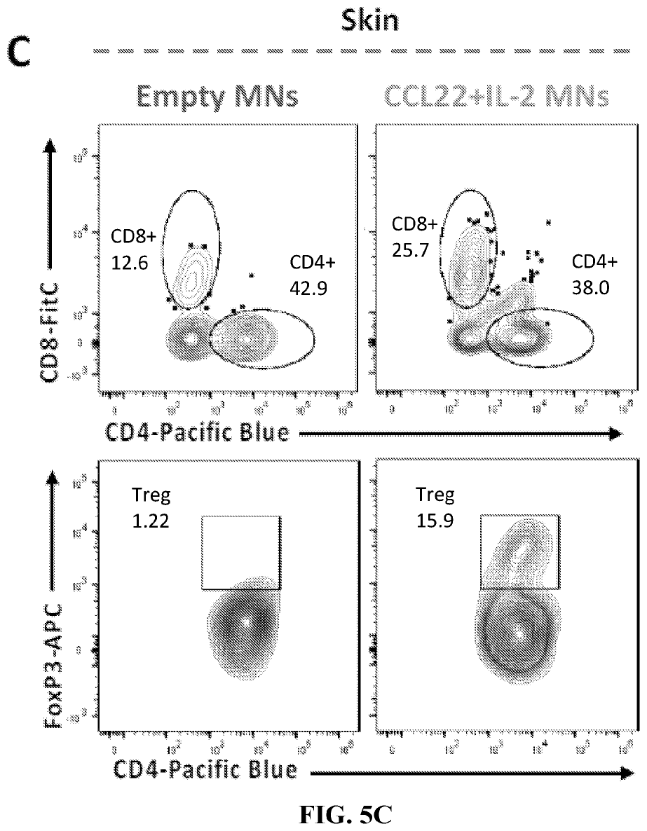

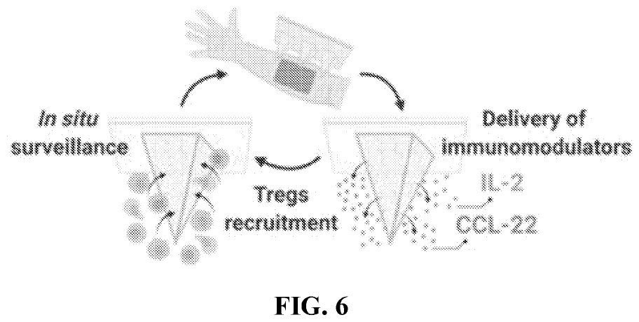

Next, the MN potential in sampling cells from the skin allograft tissue to report on transplant state following therapy was assessed. The immune cell profile was analyzed by flow cytometry. The MNs were applied and retrieved from the skin after 24 hours of application. MNs were then digested with 10 mM TCEP, centrifuged, resuspended in full media, and stained with fluorochrome-labeled antibodies for flow cytometry (FIG. 5A). Indeed, MNs that delivered CCL22 and IL-2 had higher number of T_{regs} entrapped in them compared to empty needles (FIG. 5B). To validate that this reflected the immune state in the skin allograft, the presence of T_{regs} in the allograft skin biopsy was tested by flow cytometry. Flow cytometry plots corroborated the presence of higher percentage of T_{regs} in the skin allograft treated with CCL22+IL-2 MNs compared to “Empty MN” controls, in agreement with the retrieved ISF following microneedle sampling (FIG. 5C).

### Example 8—HA-Based MNs Simultaneously Delivered Chemokines and Enabled Extraction of ISF In Vivo

In this study, a new HA-based MN was synthetized, allowing for rapid chemokines release and skin interstitial fluid (ISF) extraction, to recruit and sample T_{regs}. The MN platform was composed of amine-modified HA hydrogel crosslinked with 8-arm NHs PEG, providing high swelling capacity for a fast drug release and enhanced cell infiltration. In addition, the presence of disulfide bonds in the amine-modified HA structure allowed for its degradation, occurring in less than 5 minutes, making the MNs amenable for subsequent analysis of immune cells. In vivo studies showed that CCL22 and IL-2 loaded HA-based MNs induced T_{regs} recruitment and expansion in the skin transplant site without inducing a systemic effect. Moreover, the HA-based MNs platform allowed for sampling of the T_{regs} homing process, which can be used for the early detection of rejection episodes in skin allograft transplantation, thereby increasing the prospects of graft survival.

### Other Embodiments

It is to be understood that while certain embodiments have been described within the detailed description, the present disclosure is intended to illustrate and not limit the scope of any embodiment defined by the scope of the appended claims. Other aspects, advantages, and modifications are within the scope of the appended claims.

1.-33. (canceled)

34. A method of treating a skin disorder in a subject in need thereof, the method comprising:
contacting a microneedle array comprising a plurality of microneedles with a skin surface of the subject, wherein the plurality of microneedles comprises: i) a degradable hyaluronic acid polymer comprising a disulfide bond coupled to a terminal amine group, and ii) an immunosuppressor and/or an immunoregulator; and
applying pressure on the microneedle array such that the plurality of microneedles penetrates the skin surface, thereby releasing the immunosuppressor and/or the immunoregulator beneath the skin surface,
wherein the degradable hyaluronic acid polymer preferably comprises the following chemical structure:

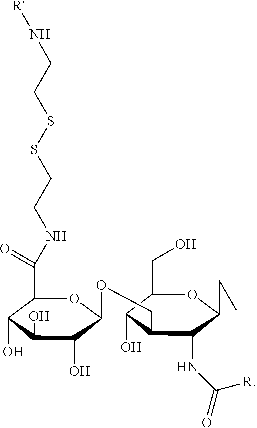

35. The method of claim 34, wherein the skin disorder is diffuse systemic scleroderma, atopic dermatitis, cutaneous lupus erythematosus, alopecia areata, alopecia totalis, alopecia universalis, androgenetic alopecia, vitiligo, psoriasis, a burn, or any combination thereof.

36. The method of claim 34, wherein the immunosuppressor and/or the immunoregulator comprises a cell, a chemokine, a cytokine, an anti-inflammatory agent, an antibody, an anti-hypertensive agent, an immunosuppressant, or any combination thereof.

37. The method of claim 36, wherein the cell is a T cell, B cell, natural killer cell, dendritic cell, macrophage, or any combination thereof.

38. The method of claim 36, wherein the chemokine comprises C-C motif chemokine 22 (CCL22), wherein the cytokine comprises IL-7, IL-2, IL-10, TGF-β, IL-33, IFN-alpha, IFN-β or any combination thereof, wherein the antibody comprises an anti-CD3 monoclonal antibody, an anti-IL-6 monoclonal antibody, an anti-CD28 monoclonal antibody, an anti-CD52 monoclonal antibody, or any combination thereof, and wherein the anti-inflammatory agent comprises a corticosteroid, a non-steroidal anti-inflammatory drug (NSAIDs), an anti-inflammatory cytokine, a cytokine antagonist, an immunosuppressant, an mTOR inhibitor, rapamycin, or any combination thereof.

39. The method of claim 34, wherein the releasing the immunosuppressor and/or the immunoregulator beneath the skin surface does not elicit a systemic response.

40. The method of claim 34, further comprising sampling an interstitial fluid using the plurality of microneedles, wherein the sampling comprises absorbing the interstitial fluid with the plurality of microneedles, and wherein the interstitial fluid comprises a biomarker and/or a cell.

41. The method claim 34, further comprising depleting an unwanted immune cell by recruiting the unwanted cells with the immunosuppressor and/or the immunoregulator.

42. The method of claim 34, wherein each microneedle of the plurality of microneedles is a degradable microneedle configured to be degraded upon exposure to a reducing agent.

43. The method of claim 42, wherein the disulfide bond is configured to be cleaved upon exposure to the reducing agent.

44. The method of claim 43, wherein the reducing agent is glutathione, dithiothreitol, or beta-mercaptoethanol.

45. The method of claim 43, wherein the reducing agent is tris(2-carboxyethyl)phosphine (TCEP), and wherein the disulfide bond is configured to be cleaved by exposure to about 1 mM to about 100 mM of TCEP.

46. The method of claim 34, wherein the hyaluronic acid polymer is a crosslinked hyaluronic acid polymer, wherein the crosslinked hyaluronic acid polymer is chemically crosslinked via a chemical crosslinker, and wherein the chemical crosslinker comprises polyethylene glycol (PEG) further comprising a succinimidyl functional group.

47. The method of claim 46, wherein the PEG is an 8-arm PEG, and wherein the PEG has a molecular weight ranging from about 10 kDa to about 200 kDa.

48. The method of claim 46, wherein the chemical crosslinker comprises a first PEG having a molecular weight of about 40 kDa and a second PEG having a molecular weight of about 10 kDa, and wherein the chemical crosslinker comprises the first PEG and the second PEG at a ratio of about 0:100 to about 100:0.

49. The method of claim 34, wherein each microneedle of the plurality of microneedles has a height of about 100 μm to about 1,500 μm and a base having a radius of about 100 μm to about 1,500 μm, and wherein each microneedle of the plurality of microneedles is a porous hydrogel.

50. The method of claim 34, wherein the plurality of microneedles project from a polymeric, biodegradable substrate, and wherein the polymeric, biodegradable substrate comprises poly(D,L-lactide-co-glycolide) polymer.

51. The method of claim 34, wherein each microneedle of the plurality of microneedles has a swelling ratio ranging from about 800% to about 1800% within at least about 15 minutes of exposure to an aqueous medium.

52. A method of locally regulating an immune response in a tissue of a subject in need thereof, the method comprising:
contacting a microneedle array comprising a plurality of microneedles with a skin surface of the subject, wherein the plurality of microneedles comprises: i) a degradable hyaluronic acid polymer comprising a disulfide bond, and ii) an immunosuppressor and/or an immunoregulator; and
applying pressure on the microneedle array such that the plurality of microneedles penetrates the skin surface, thereby releasing the immunosuppressor and/or the immunoregulator beneath the skin surface in the tissue.

53. The method of claim 52, wherein the tissue is a burned tissue, an autograft, a split-thickness skin graft, a full-thickness skin graft, an allograft, a homograft, a xenograft, a meshed graft, a sheet graft, or any combination thereof.


SEQUENCE LISTING (8 sequences)

SEQ ID NO: 1 - DNA, 23 bp, Mus musculus
  misc_feature: Synthetic primer - GAPDH forward sequence
catcactgccacccagaagactg

SEQ ID NO: 2 - DNA, 23 bp, Mus musculus
  misc_feature: Synthetic primer - GAPDH reverse sequence
atgccagtgagcttcccgttcag

SEQ ID NO: 3 - DNA, 22 bp, Mus musculus
  misc_feature: Synthetic primer - FOXP3 forward sequence
cctggttgtgagaaggtcttcg

SEQ ID NO: 4 - DNA, 22 bp, Mus musculus
  misc_feature: Synthetic primer - FOXP3 reverse sequence
tgctccagagactgcaccactt

SEQ ID NO: 5 - DNA, 22 bp, Mus musculus
  misc_feature: Synthetic primer - CD3 forward sequence
gctccaggatttctcggaagtc

SEQ ID NO: 6 - DNA, 22 bp, Mus musculus
  misc_feature: Synthetic primer - CD3 reverse sequence
atggctactgctgtcaggtcca

SEQ ID NO: 7 - DNA, 22 bp, Mus musculus
  misc_feature: Synthetic primer - IL-6 forward sequence
taccacttcacaagtcggaggc

SEQ ID NO: 8 - DNA, 23 bp, Mus musculus
  misc_feature: Synthetic primer - IL-6  reverse sequence
ctgcaagtgcatcatcgttgttc